(19) United States
(12) Patent Application Publication
(10) Pub. No.: US 2026/0224545 A1
(43) Pub. Date: Aug. 6, 2026

(54) PELLET OF DABIGATRAN ETEXILATE OR PHARMACEUTICALLY ACCEPTABLE SALT THEREOF

(71) Applicant: BOSTAL DRUG DELIVERY CO., LTD., Guangzhou (CN)

(72) Inventors: Wei Li, Guangzhou (CN); Wenxi Yan, Guangzhou (CN); Rong Liu, Guangzhou (CN); Weiqin Tong, Guangzhou (CN); Xiang Li, Guangzhou (CN)

(73) Assignee: BOSTAL DRUG DELIVERY CO., LTD., Guangzhou, GD (CN)

(21) Appl. No.: 19/158,274

(22) PCT Filed: Feb. 21, 2024
(86) PCT No.: PCT/CN2024/077952

§ 371 Date: Aug. 20, 2025

(30) Foreign Application Priority Data
Feb. 21, 2023 (CN) 202310153343.0

(51) Int. Cl. A61K 31/4439; A61K 9/16; A61K 9/20; A61K 9/28; A61K 9/48

(52) U.S. Cl. CPC A61K 31/4439; A61K 9/1694; A61K 9/2013; A61K 9/2813; A61K 9/2866; A61K 9/4808

(57) ABSTRACT

Disclosed in the present invention is a pellet of dabigatran etexilate or a pharmaceutically acceptable salt thereof, which pellet contains a pellet core, a drug layer containing dabigatran etexilate or the pharmaceutically acceptable salt thereof, and a protective layer, wherein the drug layer containing dabigatran etexilate or the pharmaceutically acceptable salt thereof is located between the pellet core and the protective layer; the protective layer contains a water-soluble polymer and a non-water-soluble cellulose derivative having a bonded hydrophobic group; and the weight ratio of the water-soluble polymer to the non-water-soluble cellulose derivative having a bonded hydrophobic group is from 0.67:1 to 1.3:1. Compared with the prior art, the present invention reduces the risk of pellet adhesion in the capsule, so as to achieve the effects of reducing the individual variation in drug administration, stabilizing the release of organic acids and drugs, improving the bioavailability, and decreasing side effects.

### CROSS REFERENCE TO RELATED APPLICATIONS

The present application claims priority to Chinese Patent Application No. 2023101533430, filed on Feb. 21, 2023, the entire contents of which are hereby incorporated by reference.

### TECHNICAL FIELD

The present invention relates to the field of medicine, and more specifically, relates to pellet of dabigatran etexilate or pharmaceutically acceptable salt thereof.

### BACKGROUND

Atrial fibrillation (abbreviated as AF) is the most common type of persistent arrhythmia. The incidence of AF continues to increase with age, reaching up to 10% in crowd aged 75 and above. During AF, the frequency of atrial excitation reaches 300 to 600 beats per minute, and the heartbeat frequency is often rapid and irregular, sometimes reaching 100 to 160 beats per minute. Not only is the heartbeat frequency much faster than a normal person heartbeat, but it is also absolutely irregular, causing the atria to lose effective contractile function.

Stroke is an acute cerebrovascular disease, which refers to a group of diseases caused by sudden rupture of cerebral blood vessels or blockage of blood vessels which prevents blood from flowing into the brain, resulting in brain tissue damage, including ischemic stroke and hemorrhagic stroke.

Embolism refers to the phenomenon where abnormal substance that are insoluble in blood appear in the circulating blood and flow with it, thereby obstructing the lumen of blood vessels.

Dabigatran etexilate mesylate is the active pharmaceutical ingredient of the commodity Pradaxa® capsule, used for the prophylactic treatment to reduce the risk of stroke and systemic embolism in patients with non-valvular atrial fibrillation. Its solubility exhibits significant pH-dependent, with higher pH values resulting in poorer solubility. In a solution with pH >5, it is almost insoluble. Dabigatran etexilate is absorbed primarily in the proximal small intestine, with a narrow drug absorption window. However, after oral administration, it often exhibits undesirable individual variability, thereby resulting in potential clinical risks. The currently marketed Pradaxa® capsule preparation was developed by Boehringer Ingelheim. Patent WO 03/074056 discloses an oral pharmaceutical combination of dabigatran etexilate, and this pharmaceutical preparation is a multilayer-coated pellet consisting of an organic acid pellet core material, an isolation layer, and an active drug layer, sequentially arranged from the inside out. The pellet is filled into capsules to obtain dabigatran etexilate capsules. The organic acid provides an acidic microenvironment for the dissolution of the active substance to achieve the goal of maintaining the same bioavailability at different pH levels. Currently, it has been found that the marketed dabigatran etexilate capsules, due to the slow dissolution of the capsule shell and the properties of the formulation excipients, exhibit issues that pellet agglomeration within the capsule under release conditions, resulting in a high relative standard deviation (RSD) in vitro release and significant individual variability in oral administration, which affects its bioavailability.

Patent CN 111840245 A discloses a dabigatran etexilate pellet, which improves the roundness of the pellet core by providing a protective layer outside the active drug layer, thereby reducing the RSD of in vitro release of dabigatran etexilate pellets. However, it still fails to address the risks brought by pellet agglomeration.

### SUMMARY OF THE INVENTION

To address the aforementioned technical problems, the present invention provides a pellet of dabigatran etexilate or a pharmaceutically acceptable salt thereof, which achieves anti-agglomeration effects through improvements to the protective layer.

In one aspect, the present invention relates to a pellet of dabigatran etexilate or a pharmaceutically acceptable salt thereof, which comprise a pellet core, a drug layer containing dabigatran etexilate or pharmaceutically acceptable salt thereof, and a protective layer. Wherein, the drug layer containing dabigatran etexilate or pharmaceutically acceptable salt thereof is located between the pellet core and the protective layer, and the protective layer comprises a water-soluble polymer and a water-insoluble cellulose derivative with bonded hydrophobic groups.

In another aspect, the present invention relates to a capsule, which comprises pellets of dabigatran etexilate or a pharmaceutically acceptable salt thereof, wherein the pellet comprise a pellet core, a drug layer containing dabigatran etexilate or pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

In yet another aspect, the present invention relates to a method for reducing individual variability of dabigatran etexilate or a pharmaceutically acceptable salt thereof, which includes administering a capsule comprising pellets of dabigatran etexilate or a pharmaceutically acceptable salt thereof, wherein the pellet comprises a pellet core, a drug layer containing dabigatran etexilate or a pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

In yet another aspect, the present invention relates to a method for reducing the risk of stroke and systemic embolism in adult patients with non-valvular atrial fibrillation, which includes administering to a patient in need thereof a prophylactically effective amount of a capsule comprising pellets of dabigatran etexilate or a pharmaceutically acceptable salt thereof, wherein the pellet comprises a pellet core, a drug layer containing dabigatran etexilate or a pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

In another aspect, the present invention relates to a method for improving the agglomeration of pellets in capsules, which includes providing the capsule with pellets of dabigatran etexilate or a pharmaceutically acceptable salt thereof, wherein the pellet comprises a pellet core, a drug layer containing dabigatran etexilate or a pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

In a further aspect, the present invention relates to a method for treating deep venous thrombosis and pulmonary embolism in adult patients, which includes administering to a patient in need thereof a prophylactically effective amount of a capsule comprising pellets of dabigatran etexilate or a pharmaceutically acceptable salt thereof, wherein the pellet comprises a pellet core, a drug layer containing dabigatran etexilate or a pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

In yet another aspect, the present invention relates to a method for reducing the recurrence risk of deep venous thrombosis and pulmonary embolism in adult patients, which includes administering to a patient in need thereof a prophylactically effective amount of a capsule comprising pellets of dabigatran etexilate or a pharmaceutically acceptable salt thereof, wherein the pellet comprises a pellet core, a drug layer containing dabigatran etexilate or a pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

In another aspect, the present invention relates to a method for preventing deep venous thrombosis and pulmonary embolism in adult patients after hip replacement surgery, which includes administering to a patient in need thereof a prophylactically effective amount of a capsule comprising pellets of dabigatran etexilate or a pharmaceutically acceptable salt thereof, wherein the pellet comprises a pellet core, a drug layer containing dabigatran etexilate or a pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

In a further aspect, the present invention relates to a method for treating venous thromboembolic events in pediatric patients, which includes administering to a patient in need thereof a prophylactically effective amount of a capsule comprising pellets of dabigatran etexilate or a pharmaceutically acceptable salt thereof, wherein the pellet comprises a pellet core, a drug layer containing dabigatran etexilate or a pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

In yet another aspect, the present invention relates to a method for reducing the recurrence risk of venous thromboembolic events in pediatric patients, which includes administering to a patient in need thereof a prophylactically effective amount of a capsule comprising pellets of dabigatran etexilate or a pharmaceutically acceptable salt thereof, wherein the pellet comprises a pellet core, a drug layer containing dabigatran etexilate or a pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

### Beneficial Effects

Compared with the prior art, the present invention reduces the risk of pellet agglomeration in a capsule, so as to achieve the effects of reducing the individual variability in drug administration, stabilizing the release of organic acids and drugs, improving the bioavailability, and decreasing side effects.

### DETAILED DESCRIPTION

In the following description, certain specific details are included to provide a comprehensive understanding of various disclosed embodiments. However, those skilled in the relevant art will recognize that embodiments can still be implemented without utilizing one or more of these specific details, and by utilizing other methods, components, materials, etc.

Unless otherwise required in the present application, throughout the specification and the appended claims, the terms “including”, “comprising”, “containing” and “having” shall be construed as having an open and inclusive meaning, that is, “including but not limited to”.

When used in the present invention and the appended claims, unless otherwise specified in the context, singular references without a quantity indication include plural references.

The phrases “one embodiment”, “an embodiment”, “in another embodiment” or “in some embodiments” mentioned throughout the entire specification refer to including specific reference elements, structures, or features related to the embodiment described in at least one embodiment. Therefore, the phrases “in one embodiment” or “in an embodiment” or “in another embodiment” or “in some embodiments” appearing at different locations throughout the entire specification do not necessarily all refer to the same embodiment. Furthermore, specific elements, structures or features may be combined in one or more embodiments in any appropriate manner.

It should be understood that the use of the singular form of the article “a” (corresponding to “a”, “an”, and “the” in English) in the specification and appended claims of the present invention includes plural objects, unless otherwise in specified in the text. Therefore, for example, the mentioned sustained-release tablet containing “a pharmaceutically acceptable excipient” includes one pharmaceutically acceptable excipient, or two or more pharmaceutically acceptable excipients.

### Definition

In the present invention, the term “dabigatran etexilate” refers to ethyl 3-[[[2-[[[4-[[[(hexyloxy)carbonyl]amino]iminomethyl]phenyl]amino]methyl]-1-methyl-1H-benzimidazol-5-yl]carbonyl](pyridin-2-yl)amino]propanoate.

In the present invention, the term “anti-adherent” refers to an agent that alleviates or improves the agglomeration between preparation units during the coating and drug-layering processes.

In the present invention, the term “water-soluble polymer” refers to hydrophilic high-molecular-weight polymer materials that can dissolve or swell in water to form aqueous solutions or dispersion systems.

In the present invention, the term “acceptable acid addition salts” refers to those salts that maintain the biological effectiveness and properties of the free base. The acid addition salt is suitable in biological or other aspects and is formed using inorganic acids or organic acids, wherein the inorganic acids such as, but not limited to, hydrochloric acid, hydrobromic acid, sulfuric acid, nitric acid, phosphoric acid, etc., the organic acids such as, but not limited to, acetic acid, 2,2-dichloroacetic acid, adipic acid, alginic acid, ascorbic acid, aspartic acid, benzenesulfonic acid, benzene carboxylic acid, 4-acetamidobenzene carboxylic acid, camphanic acid, camphor-10-sulfonic acid, decanoic acid, hexanoic acid, octanoic acid, carbonic acid, cinnamic acid, citric acid, cyclohexylsulfamic acid, dodecylsulfuric acid, ethane-1,2-disulfonic acid, ethanesulfonic acid, 2-hydroxyethanesulphonic acid, formic acid, fumaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, etc.

In the present invention, the term “acceptable base addition salts” refers to those salts that maintain the biological effectiveness and properties of the free acid, and the base addition salt is suitable in biological or other aspects. These salts are prepared by adding inorganic bases or organic bases into the free acid. Salts derived from inorganic bases include, but are not limited to, sodium, potassium, lithium, ammonium, calcium, magnesium, iron, zinc, copper, manganese, aluminum salts, etc. In some embodiments, the inorganic salts are ammonium salt, sodium salt, potassium salt, calcium salt and magnesium salt. Salts derived from organic bases include, but are not limited to, salts of primary amine, secondary amine and tertiary amine, substituted amines including naturally occurring substituted amines, cyclic amines, and salts of basic ion exchange resins, such as ammonia, isopropylamine, trimethylamine, diethylamine, triethylamine, tripropylamine, diethanolamine, ethanolamine, 2-dimethylaminoethanol, 2-diethylaminoethanol, dicyclohexylamine, lysine, arginine, histidine, caffeine, procaine, hydrabamine, choline, betaine, benzylamine, phenylethylenediamine, ethylenediamine, glucosamine, methylglucamine, theobromine, triethanolamine, tromethamine, purine, piperazine, piperidine, N-ethylpiperidine, polyamine resins, etc. In some embodiments, the organic bases are isopropylamine, diethylamine, ethanolamine, trimethylamine, dicyclohexylamine, choline and caffeine.

In the present invention, the term “individual variability” refers to the differences exhibited among homogeneous observation subjects, encompassing both inter-individual variability and intra-individual variability. Inter-individual variability refers to the differences between different administered individuals, while intra-individual variability refers to the differences generated in the same subject when taking the preparation at different time points.

In the present invention, the term “pellet” refers to a spherical or approximately spherical solid dosage form with a diameter ranging from 0.3 mm to 1.5 mm.

In the present invention, the term “water-insoluble cellulose derivative with bonded hydrophobic groups” refers to the water-insoluble products obtained through esterification or etherification of hydroxyl groups in cellulose polymer with chemical reagents, which contain hydrophobic groups as substituents.

In the present invention, the term “weight gain” refers to the percentage of weight gained during the coating process relative to the initial weight of the uncoated formulation core.

In the present invention, the weight gain of the coating is relative to the encapsulated core. Specifically, the coating weight gain of the protective layer or its components is defined relative to the weight of an encapsulated core, which consists of the tartaric acid pellet core, isolation layer and drug layer; the coating weight gain of the drug layer or its components is defined relative to the weight of an encapsulated core, which consists of the tartaric acid pellet core and the isolation layer; the coating weight gain of the isolation layer or its components is defined relative to the weight of an encapsulated core, which is the tartaric acid pellet core.

In one aspect, the present invention relates to a pellet of dabigatran etexilate or a pharmaceutically acceptable salt thereof, which comprises a pellet core, a drug layer containing dabigatran etexilate or pharmaceutically acceptable salt thereof, and a protective layer. The drug layer containing dabigatran etexilate or pharmaceutically acceptable salt thereof is located between the pellet core and the protective layer, and the protective layer comprises a water-soluble polymer and a water-insoluble cellulose derivative with bonded hydrophobic groups.

In some embodiments, the water-insoluble cellulose derivative with bonded hydrophobic groups that can be used in the protective layer of the present invention, the exemplary examples of its hydrophobic groups include, but are not limited to, methoxy, ethoxy, acetyl, butyryl and phthaloyl groups.

In some embodiments, the water-insoluble cellulose derivative with bonded hydrophobic groups that can be used in the protective layer of the present invention, when the exemplary examples of its hydrophobic groups contain methoxy, also simultaneously contain one or more other hydrophobic groups.

In some embodiments, exemplary examples of other hydrophobic groups that can be used in the present invention include, but are not limited to, ethoxy, acetyl, butyryl and phthaloyl groups; preferably one or more of ethoxy, acetyl and butyryl groups; more preferably ethoxy or acetyl group.

In some embodiments, the water-insoluble cellulose derivative with bonded hydrophobic groups that can be used in the protective layer of the present invention, wherein the content of all hydrophobic groups is about 14% to 62% by weight relative to the water-insoluble cellulose derivative with bonded hydrophobic groups.

In some embodiments, the water-insoluble cellulose derivative with bonded hydrophobic groups that can be used in the protective layer of the present invention, wherein the content of all hydrophobic groups is about 15% to 62% by weight, about 16% to 62% by weight, about 17% to 62% by weight, about 18% to 62% by weight, about 19% to 62% by weight, about 20% to 62% by weight, about 21% to 62% by weight, about 22% to 62% by weight, about 23% to 62% by weight, about 24% to 62% by weight, about 25% to 62% by weight, about 26% to 62% by weight, about 27% to 62% by weight, about 28% to 62% by weight, about 29% to 62% by weight, about 30% to 62% by weight, about 31% to 62% by weight, about 32% to 62% by weight, about 33% to 62% by weight, about 34% to 62% by weight, about 35% to 62% by weight, about 36% to 62% by weight, about 37% to 62% by weight, about 38% to 62% by weight, about 39% to 62% by weight, about 40% to 62% by weight, about 41% to 62% by weight, about 42% to 62% by weight, about 43% to 62% by weight, about 44% to 62% by weight, about 45% to 62% by weight, about 46% to 62% by weight, about 47% to 62% by weight, about 48% to 62% by weight, about 49% to 62% by weight, about 50% to 62% by weight, about 51% to 62% by weight, about 52% to 62% by weight, about 53% to 62% by weight, about 54% to 62% by weight, about 55% to 62% by weight, about 56% to 62% by weight, about 57% to 62% by weight, about 58% to 62% by weight, about 59% to 62% by weight, about 60% to 62% by weight or about 61% to 62% by weight relative to the water-insoluble cellulose derivative with bonded hydrophobic groups.

In some embodiments, the water-insoluble cellulose derivative with bonded hydrophobic groups that can be used in the protective layer of the present invention, wherein the content of the methoxy group, which acts as a hydrophobic group, is about 12% to 30% by weight relative to the water-insoluble cellulose derivative with bonded hydrophobic groups.

In some embodiments, the water-insoluble cellulose derivative with bonded hydrophobic groups that can be used in the protective layer of the present invention, wherein the content of the methoxy group, which acts as a hydrophobic group, is about 12% to 13% by weight, about 12% to 14% by weight, about 12% to 15% by weight, about 12% to 16% by weight, about 12% to 17% by weight, about 12% to 18% by weight, about 12% to 19% by weight, about 12% to 20% by weight, about 12% to 21% by weight, about 12% to 22% by weight, about 12% to 23% by weight, about 12% to 24% by weight, about 12% to 25% by weight, about 12% to 26% by weight, about 12% to 27% by weight, about 12% to 28% by weight or about 12% to 29% by weight relative to the water-insoluble cellulose derivative with bonded hydrophobic groups.

In some embodiments, the water-insoluble cellulose derivative with bonded hydrophobic groups that can be used in the protective layer of the present invention, wherein the content of the ethoxy group, which acts as a hydrophobic group, is about 28% to 55% by weight relative to the water-insoluble cellulose derivative with bonded hydrophobic groups.

In some embodiments, the water-insoluble cellulose derivative with bonded hydrophobic groups that can be used in the protective layer of the present invention, wherein the content of the ethoxy group, which acts as a hydrophobic group, is about 28% to 29% by weight, about 28% to 30% by weight, about 28% to 31% by weight, about 28% to 32% by weight, about 28% to 33% by weight, about 28% to 34% by weight, about 28% to 35% by weight, about 28% to 36% by weight, about 28% to 37% by weight, about 28% to 38% by weight, about 28% to 39% by weight, about 28% to 40% by weight, about 28% to 41% by weight, about 28% to 42% by weight, about 28% to 43% by weight, about 28% to 44% by weight, about 28% to 45% by weight, about 28% to 46% by weight, about 28% to 47% by weight, about 28% to 48% by weight, about 28% to 49% by weight, about 28% to 50% by weight, about 28% to 51% by weight, about 28% to 52% by weight, about 28% to 53% by weight or about 28% to 54% by weight relative to the water-insoluble cellulose derivative with bonded hydrophobic groups.

In some embodiments, the water-insoluble cellulose derivative with bonded hydrophobic groups that can be used in the protective layer of the present invention, wherein the content of the phthaloyl group, which acts as a hydrophobic group, is about 20% to 38% by weight relative to the water-insoluble cellulose derivative with bonded hydrophobic groups.

In some embodiments, the water-insoluble cellulose derivative with bonded hydrophobic groups that can be used in the protective layer of the present invention, wherein the content of the phthaloyl group, which acts as a hydrophobic group, is about 20% to 21% by weight, about 20% to 22% by weight, about 20% to 23% by weight, about 20% to 24% by weight, about 20% to 25% by weight, about 20% to 26% by weight, about 20% to 27% by weight, about 20% to 28% by weight, about 20% to 29% by weight, about 20% to 30% by weight, about 20% to 31% by weight, about 20% to 32% by weight, about 20% to 33% by weight, about 20% to 34% by weight, about 20% to 35% by weight, about 20% to 36% by weight, or about 20% to 37% by weight relative to the water-insoluble cellulose derivative with bonded hydrophobic groups.

In some embodiments, the water-insoluble cellulose derivative with bonded hydrophobic groups that can be used in the protective layer of the present invention, wherein the content of the acetyl group, which acts as a hydrophobic group, is about 2% to 48% by weight relative to the water-insoluble cellulose derivative with bonded hydrophobic groups.

In some embodiments, the water-insoluble cellulose derivative with bonded hydrophobic groups that can be used in the protective layer of the present invention, wherein the content of the acetyl group, which acts as a hydrophobic group, is about 2% to 3% by weight, about 2% to 4% by weight, about 2% to 5% by weight, about 2% to 6% by weight, about 2% to 7% by weight, about 2% to 8% by weight, about 2% to 9% by weight, about 2% to 10% by weight, about 2% to 11% by weight, about 2% to 12% by weight, about 2% to 13% by weight, about 2% to 14% by weight, about 2% to 15% by weight, about 2% to 16% by weight, about 2% to 17% by weight, about 2% to 18% by weight, about 2% to 19% by weight, about 2% to 20% by weight, about 2% to 21% by weight, about 2% to 22% by weight, about 2% to 23% by weight, about 2% to 24% by weight, about 2% to 25% by weight, about 2% to 26% by weight, about 2% to 27% by weight, about 2% to 28% by weight, about 2% to 29% by weight, about 2% to 30% by weight, about 2% to 31% by weight, about 2% to 32% by weight, about 2% to 33% by weight, about 2% to 34% by weight, about 2% to 35% by weight, about 2% to 36% by weight, about 2% to 37% by weight, about 2% to 38% by weight, about 2% to 39% by weight, about 2% to 40% by weight, about 2% to 41% by weight, about 2% to 42% by weight, about 2% to 43% by weight, about 2% to 44% by weight, about 2% to 45% by weight, about 2% to 46% by weight or about 2% to 47% by weight relative to the water-insoluble cellulose derivative with bonded hydrophobic groups.

In some embodiments, the water-insoluble cellulose derivative with bonded hydrophobic groups that can be used in the protective layer of the present invention, wherein the content of the butyryl group, which acts as a hydrophobic group, is about 14% to 22% by weight relative to the water-insoluble cellulose derivative with bonded hydrophobic groups.

In some embodiments, the water-insoluble cellulose derivative with bonded hydrophobic groups that can be used in the protective layer of the present invention, wherein the content of the butyryl group, which acts as a hydrophobic group, is about 14% to 15% by weight, about 14% to 16% by weight, about 14% to 17% by weight, about 14% to 18% by weight, about 14% to 19% by weight, about 14% to 20% by weight or about 14% to 21% by weight relative to the water-insoluble cellulose derivative with bonded hydrophobic groups.

In some embodiments, exemplary examples of water-insoluble cellulose derivatives with bonded hydrophobic groups that can be used in the protective layer of the present invention include, but are not limited to, cellulose acetate, cellulose acetate phthalate, cellulose acetate butyrate, ethyl cellulose, hydroxypropyl methylcellulose phthalate, hydroxypropyl methylcellulose acetate succinate and carboxymethyl ethyl cellulose.

In some embodiments, exemplary examples of water-insoluble cellulose derivatives with bonded hydrophobic groups that can be used in the protective layer of the present invention include, but are not limited to, ethyl cellulose.

In some embodiments, ethyl cellulose that can be used in the present invention has a viscosity of about 2 mPa·s to 120 mPa·s.

In some embodiments, ethyl cellulose that can be used in the present invention has a viscosity of about 2 mPa·s to 3 mPa·s, about 2 mPa·s to 4 mPa·s, about 4 mPa·s to 5 mPa·s, about 2 mPa·s to 5.5 mPa·s, about 2 mPa·s to 6 mPa·s, about 2 mPa·s to 7 mPa·s, about 2 mPa·s to 8 mPa·s, about 2 mPa·s to 9 mPa·s, about 2 mPa·s to 10 mPa·s, about 2 mPa·s to 11 mPa·s, about 2 mPa·s to 12 mPa·s, about 2 mPa·s to 13 mPa·s, about 2 mPa·s to 14 mPa·s, about 2 mPa·s to 15 mPa·s, about 2 mPa·s to 16 mPa·s, about 2 mPa·s to 17 mPa·s, about 2 mPa·s to 18 mPa·s, about 2 mPa·s to 19 mPa·s, about 2 mPa·s to 20 mPa·s, about 2 mPa·s to 21 mPa·s, about 2 mPa·s to 22 mPa·s, about 2 mPa·s to 23 mPa·s, about 2 mPa·s to 24 mPa·s, about 2 mPa·s to 25 mPa·s, about 2 mPa·s to 26 mPa·s, about 2 mPa·s to 27 mPa·s, about 2 mPa·s to 28 mPa·s, about 2 mPa·s to 29 mPa·s, about 2 mPa·s to 30 mPa·s, about 2 mPa·s to 32 mPa·s, about 2 mPa·s to 34 mPa·s, about 2 mPa·s to 36 mPa·s, about 2 mPa·s to 38 mPa·s, about 2 mPa·s to 40 mPa·s, about 2 mPa·s to 42 mPa·s, about 2 mPa·s to 44 mPa·s, about 2 mPa·s to 46 mPa·s, about 2 mPa·s to 48 mPa·s, about 2 mPa·s to 50 mPa·s, about 2 mPa·s to 60 mPa·s, about 2 mPa·s to 70 mPa·s, about 2 mPa·s to 80 mPa·s, about 2 mPa·s to 90 mPa·s, about 25 is 2 mPa·s to 100 mPa·s or about 2 mPa·s to 110 mPa·s.

In some embodiments, the molecular weight of ethyl cellulose that can be used in the present invention is about 30,000 g/mol to 220,000 g/mol.

In some embodiments, the molecular weight of ethyl cellulose that can be used in the present invention is about 30,000 g/mol to 35,000 g/mol, about 30,000 g/mol to 40,000 g/mol, about 30,000 g/mol to 45,000 g/mol, about 30,000 g/mol to 50,000 g/mol, about 30,000 g/mol to 55,000 g/mol, about 30,000 g/mol to 60,000 g/mol, about 30,000 g/mol to 65,000 g/mol, about 30,000 g/mol to 70,000 g/mol, about 30,000 g/mol to 75,000 g/mol, about 30,000 g/mol to 80,000 g/mol, about 30,000 g/mol to 85,000 g/mol, about 30,000 g/mol to 90,000 g/mol, about 30,000 g/mol to 95,000 g/mol, about 30,000 g/mol to 100,000 g/mol, about 30,000 g/mol to 105,000 g/mol, about 30,000 g/mol to 110,000 g/mol, about 30,000 g/mol to 115,000 g/mol, about 30,000 g/mol to 120,000 g/mol, about 30,000 g/mol to 125,000 g/mol, about 30,000 g/mol to 130,000 g/mol, about 30,000 g/mol to 135,000 g/mol, about 30,000 g/mol to 140,000 g/mol, about 30,000 g/mol to 145,000 g/mol, about 30,000 g/mol to 145,000 g/mol, about 30,000 g/mol to 150,000 g/mol, about 30,000 g/mol to 155,000 g/mol, about 30,000 g/mol to 160,000 g/mol, about 30,000 g/mol to 165,000 g/mol, about 30,000 g/mol to 170,000 g/mol, about 30,000 g/mol to 175,000 g/mol, about 30,000 g/mol to 180,000 g/mol, about 30,000 g/mol to 190,000 g/mol, about 30,000 g/mol to 195,000 g/mol, about 30,000 g/mol to 200,000 g/mol, about 30,000 g/mol to 205,000 g/mol, about 30,000 g/mol to 210,000 g/mol or about 30,000 g/mol to 215,000 g/mol.

In some embodiments, exemplary examples of water-insoluble cellulose derivatives with bonded hydrophobic groups that can be used in the protective layer of the present invention include, but are not limited to, cellulose acetate.

In some embodiments, the cellulose acetate that can be used in the present invention has a viscosity of about 120 mPa·s to 4,500 mPa·s, about 120 mPa·s to 3,800 mPa·s, about 120 mPa·s to 3,900 mPa·s, about 120 mPa·s to 4,000 mPa·s, about 120 mPa·s to 4,100 mPa·s, about 120 mPa·s to 4,200 mPa·s, about 120 mPa·s to 4,300 mPa·s or about 120 mPa·s to 4,400 mPa·s, as measured according to ASTMD1343 and D871 measurement methods.

In some embodiments, the cellulose acetate that can be used in the present invention has a viscosity of about 120 mPa·s to 330 mPa·s, about 130 mPa·s to 320 mPa·s, about 140 mPa·s to 310 mPa·s, about 150 mPa·s to 300 mPa·s, about 160 mPa·s to 290 mPa·s or about 170 mPa·s to 280 mPa·s, as measured according to ASTMD1343 and D871 measurement methods.

In some embodiments, the cellulose acetate that can be used in the present invention has a viscosity of about 3,000 mPa s to 4,600 mPa·s, about 3,100 mPa·s to 4,500 mPa·s, about 3,200 mPa·s to 4,400 mPa·s, about 3,300 mPa·s to 4,300 mPa·s, about 3,400 mPa·s to 4,200 mPa·s, about 3,500 mPa·s to 4,100 mPa·s, about 3,600 mPa·s to 4,000 mPa·s or about 3,700 mPa·s to 3,900 mPa·s, as measured according to ASTMD1343 and D871 measurement methods.

In some embodiments, the molecular weight of cellulose acetate that can be used in the present invention is about 10,000 g/mol to 45,000 g/mol, about 11,000 g/mol to 44,000 g/mol, about 12,000 g/mol to 43,000 g/mol, about 13,000 g/mol to 42,000 g/mol, about 14,000 g/mol to 41,000 g/mol, about 15,000 g/mol to 40,000 g/mol, about 16,000 g/mol to 39,000 g/mol, about 17,000 g/mol to 38,000 g/mol or about 18,000 g/mol to 37,000 g/mol.

In some embodiments, exemplary examples of water-insoluble cellulose derivatives with bonded hydrophobic groups that can be used in the protective layer of the present invention include, but are not limited to, cellulose acetate phthalate.

In some embodiments, the cellulose acetate phthalate that can be used in the present invention has a viscosity of about 35 mPa·s to 100 mPa·s, about 36 mPa·s to 99 mPa·s, about 37 mPa·s to 98 mPa·s, about 38 mPa·s to 97 mPa·s, about 39 mPa·s to 96 mPa·s, about 40 mPa·s to 95 mPa·s, about 41 mPa·s to 94 mPa·s, about 42 mPa·s to 93 mPa·s, about 43 mPa·s to 92 mPa·s, about 44 mPa·s to 91 mPa·s or about 45 mPa·s to 90 mPa·s, as measured using an Ubbelohde viscometer.

In some embodiments, the molecular weight of cellulose acetate phthalate that can be used in the present invention is about 8,000 g/mol to 18,000 g/mol, about 9,000 g/mol to 17,000 g/mol, about 10,000 g/mol to 16,000 g/mol, about 11,000 g/mol to 15,000 g/mol or about 12,000 g/mol to 14,000 g/mol.

In some embodiments, exemplary examples of water-insoluble cellulose derivatives with bonded hydrophobic groups that can be used in the protective layer of the present invention include, but are not limited to, cellulose acetate butyrate.

In some embodiments, the cellulose acetate butyrate that can be used in the present invention has a viscosity of about 4,337 mPa·s to 7,137 mPa·s, about 4,537 mPa·s to 6,937 mPa·s, about 4,737 mPa·s to 6,737 mPa·s, about 4,937 mPa·s to 6,537 mPa·s, about 5,137 mPa·s to 6,337 mPa·s, about 5,337 mPa·s to 6,137 mPa·s or about 5,537 mPa·s to 5,937 mPa·s, as measured according to ASTMD1343 and D817 measurement methods.

In some embodiments, the molecular weight of cellulose acetate butyrate that can be used in the present invention is about 60,000 g/mol to 70,000 g/mol, about 61,000 g/mol to 69,000 g/mol, about 62,000 g/mol to 68,000 g/mol, about 63,000 g/mol to 67,000 g/mol or about 64,000 g/mol to 66,000 g/mol.

In some embodiments, exemplary examples of water-insoluble cellulose derivative with bonded hydrophobic groups that can be used in the protective layer of the present invention include, but are not limited to, hydroxypropyl methylcellulose phthalate. In some embodiments, the hydroxypropyl methylcellulose phthalate that can be used in the present invention has a viscosity of about 20 mPa·s to 230 mPa·s, about 20 mPa·s to 25 mPa·s, about 20 mPa·s to 30 mPa·s, about 20 mPa·s to 35 mPa·s, about 20 mPa·s to 40 mPa·s, about 20 mPa·s to 45 mPa·s, about 20 mPa·s to 50 mPa·s, about 20 mPa·s to 55 mPa·s, about 20 mPa·s to 60 mPa·s, about 20 mPa·s to 65 mPa·s, about 20 mPa·s to 70 mPa·s, about 20 mPa·s to 75 mPa·s, about 20 mPa·s to 80 mPa·s, about 20 mPa·s to 85 mPa·s, about 20 mPa·s to 90 mPa·s, about 20 mPa·s to 95 mPa·s, about 20 mPa·s to 100 mPa·s, about 20 mPa·s to 105 mPa·s, about 20 mPa·s to 110 mPa·s, about 20 mPa·s to 115 mPa·s, about 20 mPa·s to 120 mPa·s, about 20 mPa·s to 125 mPa·s, about 20 mPa·s to 130 mPa·s, about 20 mPa·s to 135 mPa·s, about 20 mPa·s to 140 mPa·s, about 20 mPa·s to 145 mPa·s, about 20 mPa·s to 150 mPa·s, about 20 mPa·s to 155 mPa·s, about 20 mPa·s to 160 mPa·s, about 20 mPa·s to 165 mPa·s, about 20 mPa·s to 170 mPa·s, about 20 mPa·s to 175 mPa·s, about 20 mPa·s to 180 mPa·s, about 20 mPa·s to 185 mPa·s, about 20 mPa·s to 190 mPa·s, about 20 mPa·s to 195 mPa·s, about 20 mPa·s to 200 mPa·s, about 20 mPa·s to 205 mPa·s, about 20 mPa·s to 210 mPa·s, about 20 mPa·s to 215 mPa·s, about 20 mPa·s to 220 mPa·s and about 20 mPa·s to 225 mPa·s.

In some embodiments, the molecular weight of hydroxypropyl methylcellulose phthalate that can be used in the present invention is about 30,000 g/mol to 160,000 g/mol, about 30,000 g/mol to 40,000 g/mol, about 30,000 g/mol to 50,000 g/mol, about 30,000 g/mol to 60,000 g/mol, about 30,000 g/mol to 70,000 g/mol, about 30,000 g/mol to 80,000 g/mol, about 30,000 g/mol to 90,000 g/mol, about 30,000 g/mol to 100,000 g/mol, about 30,000 g/mol to 110,000 g/mol, about 30,000 g/mol to 120,000 g/mol, about 30,000 g/mol to 130,000 g/mol, about 30,000 g/mol to 140,000 g/mol or about 30,000 g/mol to 150,000 g/mol.

In some embodiments, exemplary examples of water-insoluble cellulose derivative with bonded hydrophobic groups that can be used in the protective layer of the present invention include, but are not limited to, hydroxypropyl methylcellulose acetate succinate.

In some embodiments, the hydroxypropyl methylcellulose acetate succinate that can be used in the present invention has a viscosity of about 1.5 mPa·s to 10 mPa·s, about 1.5 mPa·s to 1.6 mPa·s, about 1.5 mPa·s to 1.8 mPa·s, about 1.5 mPa·s to 2 mPa·s, about 1.5 mPa·s to 2.2 mPa·s, about 1.5 mPa·s to 2.4 mPa·s, about 1.5 mPa·s to 2.6 mPa·s, about 1.5 mPa·s to 2.8 mPa·s, about 1.5 mPa·s to 3.0 mPa·s, about 1.5 mPa·s to 3.2 mPa·s, about 1.5 mPa·s to 3.4 mPa·s, about 1.5 mPa·s to 3.6 mPa·s, about 1.5 mPa·s to 3.8 mPa·s, about 1.5 mPa·s to 4 mPa·s, about 1.5 mPa·s to 4.5 mPa·s, about 1.5 mPa·s to 5 mPa·s, about 1.5 mPa·s to 5.5 mPa·s, about 1.5 mPa·s to 6 mPa·s, about 1.5 mPa·s to 6.5 mPa·s, about 1.5 mPa·s to 7 mPa·s, about 1.5 mPa·s to 7.5 mPa·s, about 1.5 mPa·s to 8 mPa·s, about 1.5 mPa·s to 8.5 mPa·s, about 1.5 mPa·s to 9 mPa·s or about 1.5 mPa·s to 9.5 mPa·s.

In some embodiments, the molecular weight of hydroxypropyl methylcellulose acetate succinate that can be used in the present invention is about 14,500 g/mol to 23,500 g/mol, about 14,500 g/mol to 15,000 g/mol, about 14,500 g/mol to 15,500 g/mol, about 14,500 g/mol to 16,000 g/mol, about 14,500 g/mol to 16,500 g/mol, about 14,500 g/mol to 17,000 g/mol, about 14,500 g/mol to 17,500 g/mol, about 14,500 g/mol to 18,000 g/mol, about 14,500 g/mol to 18,500 g/mol, about 14,500 g/mol to 19,000 g/mol, about 14,500 g/mol to 19,500 g/mol, about 14,500 g/mol to 20,000 g/mol, about 14,500 g/mol to 20,500 g/mol, about 14,500 g/mol to 21,000 g/mol, about 14,500 g/mol to 21,500 g/mol, about 14,500 g/mol to 22,000 g/mol, about 14,500 g/mol to 22,500 g/mol or about 14,500 g/mol to 23,000 g/mol.

In some embodiments, exemplary examples of water-insoluble cellulose derivative with bonded hydrophobic groups that can be used in the protective layer of the present invention include, but are not limited to, carboxymethyl ethyl cellulose.

In some embodiments, the carboxymethyl ethyl cellulose that can be used in the present invention has a viscosity of about 5 mm²/s to 85 mm²/s, about 10 mm²/s to 80 mm²/s, about 15 mm²/s to 75 mm²/s or about 20 mm²/s to 70 mm²/s.

In some embodiments, the molecular weight of carboxymethyl ethyl cellulose that can be used in the present invention is about 44,000 g/mol to 54,000 g/mol, about 45,000 g/mol to 53,000 g/mol, about 46,000 g/mol to 52,000 g/mol, about 47,000 g/mol to 51,000 g/mol or about 48,000 g/mol to 50,000 g/mol.

In some embodiments, the weight ratio of the water-soluble polymer to the water-insoluble cellulose derivative with bonded hydrophobic groups in the protective layer of the pellets of the present invention is about 16:1 to 1:8. In some embodiments, the weight ratio of the water-soluble polymer to the water-insoluble cellulose derivative with bonded hydrophobic groups in the protective layer of the pellets of the present invention is about 15:1 to 1:8, about 14:1 to 1:8, about 13:1 to 1:8, about 12:1 to 1:8, about 11:1 to 1:8, about 10:1 to 1:8, about 9:1 to 1:8, about 8:1 to 1:8, about 7:1 to 1:8, about 6:1 to 1:8, about 5:1 to 1:8, about 4:1 to 1:8, about 3:1 to 1:8, about 2:1 to 1:8 or about 1:1 to 1:8.

In some embodiments, the weight ratio of the water-soluble polymer to the water-insoluble cellulose derivative with bonded hydrophobic groups in the protective layer of the pellets of the present invention is about 16:1 to 1:7, about 16:1 to 1:6, about 16:1 to 1:5, about 16:1 to 1:4, about 16:1 to 1:3, about 16:1 to 1:2 or about 16:1 to 1:1.

In some embodiments, the weight ratio of the water-soluble polymer to the water-insoluble cellulose derivative with bonded hydrophobic groups in the protective layer of the pellets of the present invention is about 8:1 to 1:4 or about 4:1 to 1:2.

In some embodiments, the coating weight gain of the protective layer of the pellets of the present invention relative to the weight of encapsulated core is about 0.1% to 12%.

In some embodiments, the coating weight gain of the protective layer of the pellets of the present invention relative to the weight of encapsulated core is about 0.6% to 12%, about 1.1% to 12%, about 1.6% to 12%, about 2.1% to 12%, about 2.6% to 12%, about 3.1% to 12%, about 3.6% to 12%, about 4.1% to 12%, about 4.6% to 12%, about 5.1% to 12%, about 5.6% to 12%, about 6.1% to 12%, about 6.6% to 12%, about 7.1% to 12%, about 7.6% to 12%, about 8.1% to 12%, about 8.6% to 12%, about 9.1% to 12%, about 9.6% to 12%, about 10.1% to 12%, about 10.6% to 12%, about 11.1% to 12% or about 11.6% to 12%.

In some embodiments, the coating weight gain of the protective layer of the pellets of the present invention relative to the weight of encapsulated core is about 1% to 8%.

In some embodiments, the coating weight gain of the protective layer of the pellets of the present invention relative to the weight of encapsulated core is about 2% to 5%.

In some embodiments, the weight of the water-insoluble cellulose derivative with bonded hydrophobic groups in the protective layer of the pellets of the present invention is about 0.1% to 8% relative to the weight of encapsulated core.

In some embodiments, the weight of the water-insoluble cellulose derivative with bonded hydrophobic groups in the protective layer of the pellets of the present invention is about 0.3% to 8%, about 0.3% to 0.5%, about 0.3% to 0.7%, about 0.3% to 0.9%, about 0.3% to 1.1%, about 0.3% to 1.3%, about 0.3% to 1.5%, about 0.3% to 1.7%, about 0.3% to 1.9%, about 0.3% to 2.1%, about 0.3% to 2.3%, about 0.3% to 2.5%, about 0.3% to 2.7%, about 0.3% to 2.9%, about 0.3% to 3.1%, about 0.3% to 3.3%, about 0.3% to 3.5%, about 0.3% to 3.7%, about 0.3% to 3.9%, about 0.3% to 4.1%, about 0.3% to 4.3%, about 0.3% to 4.5%, about 0.3% to 4.7%, 0.3% to 4.9%, about 0.3% to 5.1%, about 0.3% to 5.3%, about 0.3% to 5.5%, about 0.3% to 5.7%, about 0.3% to 5.9%, about 0.3% to 6.1%, about 0.3% to 6.3%, about 0.3% to 6.5%, about 0.3% to 6.7%, about 0.3% to 6.9%, about 0.3% to 7.1%, about 0.3% to 7.3%, about 0.3% to 7.5%, about 0.3% to 7.7% or about 0.3% to 7.9% relative to the weight of encapsulated core.

In some embodiments, the weight of the water-insoluble cellulose derivative with bonded hydrophobic groups in the protective layer of the pellets of the present invention is about 0.4% to 4% relative to the weight of encapsulated core.

In some embodiments, the weight of the water-insoluble cellulose derivative with bonded hydrophobic groups in the protective layer of the pellets of the present invention is about 0.5% to 3% relative to the weight of encapsulated core.

In some embodiments, exemplary examples of water-soluble polymers that can be used in the protective layer of the present invention include, but are not limited to, hydroxypropyl cellulose, hydroxypropyl methyl cellulose, polyvinylpyrrolidone, hydroxymethyl cellulose, hydroxyethyl cellulose and carboxymethyl cellulose; preferably from hydroxypropyl cellulose and hydroxypropyl methyl cellulose.

In some preferred embodiments, the weight ratio of the water-soluble polymer to the water-insoluble cellulose derivative with bonded hydrophobic groups in the protective layer of the pellets of the present invention ranges from 0.67:1 to 1.3:1, and the water-insoluble cellulose derivative with bonded hydrophobic groups is ethyl cellulose. When the weight ratio of the water-soluble polymer to the water-insoluble cellulose derivative with bonded hydrophobic groups is greater than 1.3:1, pellet agglomeration occurs; when the weight ratio is less than 0.67:1, the in vitro release of the pellets is slow. In other preferred embodiments, the weight ratio of the water-soluble polymer to the water-insoluble cellulose derivative with bonded hydrophobic groups ranges from 0.67:1 to 0.84:1, 0.67:1 to 1:1, 0.67:1 to 1.2:1, 0.84:1 to 1:1, 0.84:1 to 1.2:1, 0.84:1 to 1.3:1, 1:1 to 1.2:1, 1:1 to 1.3:1 or 1.2:1 to 1.3:1.

In some preferred embodiments, the coating weight gain of the protective layer of the pellets of the present invention relative to the weight of encapsulated core is about 1% to 5%. When the weight gain of the protective layer relative to the coating of the encapsulated core is 0.44%, pellet agglomeration occurs. When the weight gain of the coating is 6.1%, the in vitro release of the pellets is slow. In other preferred embodiments, the coating weight gain of the protective layer relative to the weight of encapsulated core is about 1% to 4.8%, about 1% to 4.7%, about 1% to 3.8%, about 1% to 3.6%, about 1% to 3.3%, about 1% to 3.2%, about 1% to 3.1%, about 1% to 3.0%, about 1% to 2.9%, about 1% to 2.81%, about 1% to 2.5%, about 1% to 2.2%, about 1% to 2.1%, about 1% to 1.9%, about 1% to 1.56%, about 1% to 1.08%, about 1.08% to 4.8%, about 1.08% to 4.7%, about 1.08% to 3.8%, about 1.08% to 3.6%, about 1.08% to 3.3%, about 1.08% to 3.2%, about 1.08% to 3.1%, about 1.08% to 3.0%, about 1.08% to 2.9%, about 1.08% to 2.81%, about 1.08% to 2.5%, about 1.08% to 2.2%, about 1.08% to 2.1%, about 1.08% to 1.9%, about 1.08% to 1.56%, about 1.56% to 4.8%, about 1.56% to 4.7%, about 1.56% to 3.8%, about 1.56% to 3.6%, about 1.56% to 3.3%, about 1.56% to 3.2%, about 1.56% to 3.1%, about 1.56% to 3.0%, about 1.56% to 2.9%, about 1.56% to 2.81%, about 1.56% to 2.5%, about 1.56% to 2.2%, about 1.56% to 2.1%, about 1.56% to 1.9%, about 1.9% to 4.8%, about 1.9% to 4.7%, about 1.9% to 3.8%, about 1.9% to 3.6%, about 1.9% to 3.3%, about 1.9% to 3.2%, about 1.9% to 3.1%, about 1.9% to 3.0%, about 1.9% to 2.9%, about 1.9% to 2.81%, about 1.9% to 2.5%, about 1.9% to 2.2%, about 1.9% to 2.1%, about 2.1% to 4.8%, about 2.1% to 4.7%, about 2.1% to 3.8%, about 2.1% to 3.6%, about 2.1% to 3.3%, about 2.1% to 3.2%, about 2.1% to 3.1%, about 2.1% to 3.0%, about 2.1% to 2.9%, about 2.1% to 2.81%, about 2.1% to 2.2%, about 2.2% to 4.8%, about 2.2% to 4.7%, about 2.2% to 3.8%, about 2.2% to 3.6%, about 2.2% to 3.3%, about 2.2% to 3.2%, about 2.2% to 3.1%, about 2.2% to 3.0%, about 2.2% to 2.9%, about 2.2% to 2.81%, about 2.2% to 2.5%, about 2.5% to 4.8%, about 2.5% to 4.7%, about 2.5% to 3.8%, about 2.5% to 3.6%, about 2.5% to 3.3%, about 2.5% to 3.2%, about 2.5% to 3.1%, about 2.5% to 3.0%, about 2.5% to 2.9%, about 2.5% to 2.81%, about 2.81% to 4.8%, about 2.81% to 4.7%, about 2.81% to 3.8%, about 2.81% to 3.6%, about 2.81% to 3.3%, about 2.81% to 3.2%, about 2.81% to 3.1%, about 2.81% to 3.0%, about 2.81% to 2.9%, about 2.9% to 4.8%, about 2.9% to 4.7%, about 2.9% to 3.8%, about 2.9% to 3.6%, about 2.9% to 3.3%, about 2.9% to 3.2%, about 2.9% to 3.1%, about 2.9% to 3.0%, about 3.0% to 4.8%, about 3.0% to 4.7%, about 3.0% to 3.8%, about 3.0% to 3.6%, about 3.0% to 3.3%, about 3.0% to 3.2%, about 3.0% to 3.1%, about 3.1% to 4.8%, about 3.1% to 4.7%, about 3.1% to 3.8%, about 3.1% to 3.6%, about 3.1% to 3.3%, about 3.1% to 3.2%, about 3.2% to 4.8%, about 3.2% to 4.7%, about 3.2% to 3.8%, about 3.2% to 3.6%, about 3.2% to 3.3%, about 3.3% to 4.8%, about 3.3% to 4.7%, about 3.3% to 3.8%, about 3.3% to 3.6%, about 3.6% to 4.7%, about 3.6% to 3.8%, about 3.8% to 4.8%, about 3.8% to 4.7%, about 4.7% to 4.8%, about 4.8% to 5%.

In some preferred embodiments, the weight gain of ethyl cellulose in the protective layer of the pellets of the present invention relative to the weight of encapsulated core is about 0.47% to 2.50%. When the weight gain of ethyl cellulose relative to the weight of encapsulated core is 0.19%, pellet agglomeration occurs; when the weight gain is 2.63%, the in vitro release of the pellets is slow. In other preferred embodiments, the weight gain of the ethyl cellulose relative to the weight of encapsulated core is about 0.47% to 2.48%, about 0.47% to 2.03%, about 0.47% to 1.76%, about 0.47% to 1.64%, about 0.47% to 1.55%, about 0.47% to 1.50%, about 0.47% to 1.47%, about 0.47% to 1.43%, about 0.47% to 1.34%, about 0.47% to 1.20%, about 0.47% to 0.99%, about 0.47% to 0.91%, about 0.47% to 0.82%, about 0.47% to 0.67%, about 0.67% to 2.50%, about 0.67% to 2.48%, about 0.67% to 2.03%, about 0.67% to 1.76%, about 0.67% to 1.64%, about 0.67% to 1.55%, about 0.67% to 1.50%, about 0.67% to 1.47%, about 0.67% to 1.43%, about 0.67% to 1.34%, about 0.67% to 1.20%, about 0.67% to 0.99%, about 0.67% to 0.91%, about 0.67% to 0.82%, about 0.82% to 2.50%, about 0.82% to 2.48%, about 0.82% to 2.03%, about 0.82% to 1.76%, about 0.82% to 1.64%, about 0.82% to 1.55%, about 0.82% to 1.50%, about 0.82% to 1.47%, about 0.82% to 1.43%, about 0.82% to 1.34%, about 0.82% to 1.2%, about 0.82% to 0.99%, about 0.82% to 0.91%, about 0.91% to 2.50%, about 0.91% to 2.48%, about 0.91% to 2.03%, about 0.91% to 1.76%, about 0.91% to 1.64%, about 0.91% to 1.55%, about 0.91% to 1.50%, about 0.91% to 1.47%, about 0.91% to 1.43%, about 0.91% to 1.34%, about 0.91% to 1.20%, about 0.91% to 0.99%, about 0.99% to 2.50%, about 0.99% to 2.48%, about 0.99% to 2.03%, about 0.99% to 1.76%, about 0.99% to 1.76%, about 0.99% to 1.64%, about 0.99% to 1.55%, about 0.99% to 1.50%, about 0.99% to 1.47%, about 0.99% to 1.43%, about 0.99% to 1.34%, about 0.99% to 1.20%, about 1.20% to 2.50%, about 1.20% to 2.48%, about 1.20% to 2.03%, about 1.20% to 1.76%, about 1.20% to 1.64%, about 1.20% to 1.55%, about 1.20% to 1.50%, about 1.20% to 1.47%, about 1.20% to 1.43%, about 1.20% to 1.34%, about 1.34% to 2.50%, about 1.34% to 2.48%, about 1.34% to 2.03%, about 1.34% to 1.76%, about 1.34% to 1.64%, about 1.34% to 1.55%, about 1.34% to 1.50%, about 1.34% to 1.47%, about 1.34% to 1.43%, about 1.43% to 2.50%, about 1.43% to 2.48%, about 1.43% to 2.03%, about 1.43% to 1.76%, about 1.43% to 1.64%, about 1.43% to 1.55%, about 1.43% to 1.50%, about 1.43% to 1.47%, about 1.47% to 2.50%, about 1.47% to 2.48%, about 1.47% to 2.03%, about 1.47% to 1.76%, about 1.47% to 1.64%, about 1.47% to 1.55%, about 1.47% to 1.50%, about 1.50% to 2.50%, about 1.50% to 2.48%, about 1.50% to 2.03%, about 1.50% to 1.76%, about 1.50% to 1.64%, about 1.50% to 1.55%, about 1.55% to 2.50%, about 1.55% to 2.48%, about 1.55% to 2.03%, about 1.55% to 1.76%, about 1.55% to 1.64%, about 1.64% to 2.50%, about 1.64% to 2.48%, about 1.64% to 2.03%, about 1.64% to 1.76%, about 1.76% to 2.50%, about 1.76% to 2.48%, about 1.76% to 2.03%, about 2.03% to 2.50%, about 2.03% to 2.48%, about 2.48% to 2.50%.

In some preferred embodiments, the weight gain of the water-soluble polymer in the protective layer of the pellets of the present invention relative to the weight of encapsulated core is about 0.56% to 2.44%. When the weight gain of the water-soluble polymer relative to the weight of encapsulated core is 0.23%, pellet agglomeration occurs; when the weight gain is 3.16%, the in vitro release of the pellets is slow. In other preferred embodiments, the weight gain of the water-soluble polymer relative to the weight of encapsulated core is about 0.56% to 2.08%, about 0.56% to 1.97%, about 0.56% to 1.87%, about 0.56% to 1.71%, about 0.56% to 1.61%, about 0.56% to 1.56%, about 0.56% to 1.47%, about 0.56% to 1.26%, about 0.56% to 1.19%, about 0.56% to 1.18%, about 0.56% to 1.09%, about 0.56% to 0.98%, about 0.56% to 0.84%, about 0.56% to 0.81%, about 0.81% to 2.44%, about 0.81% to 2.08%, about 0.81% to 1.97%, about 0.81% to 1.87%, about 0.81% to 1.71%, about 0.81% to 1.61%, about 0.81% to 1.56%, about 0.81% to 1.47%, about 0.81% to 1.26%, about 0.81% to 1.19%, about 0.81% to 1.18%, about 0.81% to 1.09%, about 0.81% to 0.98%, about 0.81% to 0.84%, about 0.84% to 2.44%, about 0.84% to 2.08%, about 0.84% to 1.97%, about 0.84% to 1.87%, about 0.84% to 1.71%, about 0.84% to 1.61%, about 0.84% to 1.56%, about 0.84% to 1.47%, about 0.84% to 1.26%, about 0.84% to 1.19%, about 0.84% to 1.18%, about 0.84% to 1.09%, about 0.84% to 1.08%, about 0.84% to 0.98%, about 0.98% to 2.44%, about 0.98% to 2.08%, about 0.98% to 1.97%, about 0.98% to 1.87%, about 0.98% to 1.71%, about 0.98% to 1.61%, about 0.98% to 1.56%, about 0.98% to 1.47%, about 0.98% to 1.26%, about 0.98% to 1.19%, about 0.98% to 1.18%, about 0.98% to 1.09%, about 1.09% to 2.44%, about 1.09% to 2.08%, about 1.09% to 1.97%, about 1.09% to 1.87%, about 1.09% to 1.71%, about 1.09% to 1.61%, about 1.09% to 1.56%, about 1.09% to 1.47%, about 1.09% to 1.26%, about 1.09% to 1.19%, about 1.09% to 1.18%, about 1.18% to 2.44%, about 1.18% to 2.08%, about 1.18% to 1.97%, about 1.18% to 1.87%, about 1.18% to 1.71%, about 1.18% to 1.61%, about 1.18% to 1.56%, about 1.18% to 1.47%, about 1.18% to 1.26%, about 1.18% to 1.19%, about 1.19% to 2.44%, about 1.19% to 2.08%, about 1.19% to 1.97%, about 1.19% to 1.87%, about 1.19% to 1.71%, about 1.19% to 1.61%, about 1.19% to 1.56%, about 1.19% to 1.47%, about 1.19% to 1.26%, about 1.26% to 2.44%, about 1.26% to 2.08%, about 1.26% to 1.97%, about 1.26% to 1.87%, about 1.26% to 1.71%, about 1.26% to 1.61%, about 1.26% to 1.56%, about 1.26% to 1.47%, about 1.47% to 2.44%, about 1.47% to 2.08%, about 1.47% to 1.97%, about 1.47% to 1.87%, about 1.47% to 1.71%, about 1.47% to 1.61%, about 1.47% to 1.56%, about 1.56% to 2.44%, about 1.56% to 2.08%, about 1.56% to 1.97%, about 1.56% to 1.87%, about 1.56% to 1.71%, about 1.56% to 1.61%, about 1.61% to 2.44%, about 1.61% to 2.08%, about 1.61% to 1.97%, about 1.61% to 1.87%, about 1.61% to 1.71%, about 1.71% to 2.44%, about 1.71% to 2.08%, about 1.71% to 1.97%, about 1.71% to 1.87%, about 1.87% to 2.44%, about 1.87% to 2.08%, about 1.87% to 1.97%, about 1.97% to 2.44%, about 1.97% to 2.08%, about 2.08% to 2.44%.

In some embodiments, the protective layer in the pellets of the present invention further comprises an anti-adherent. In some embodiments, exemplary examples of anti-adherent that can be used in the protective layer of the present invention include, but are not limited to, talc, colloidal silicon dioxide, titanium dioxide, magnesium stearate, stearic acid and glyceryl monostearate.

In some embodiments, the pellets of the present invention further comprise an isolation layer, wherein the isolation layer is located between the pellet core and the drug layer.

In some embodiments, the isolation layer of the pellets of the present invention comprises a water-soluble polymer and optionally a water-insoluble cellulose derivative with bonded hydrophobic groups.

In some embodiments, the isolation layer in the pellets of the present invention comprises a water-soluble polymer and optionally anti-adherent.

In some embodiments, exemplary examples of anti-adherent that can be used in the isolation layer of the present invention include, but are not limited to, talc, colloidal silicon dioxide, titanium dioxide, magnesium stearate, stearic acid and glyceryl monostearate. In some embodiments, exemplary examples of water-soluble polymers that can be used in the isolation layer of the present invention include, but are not limited to, hydroxypropyl cellulose, hydroxypropyl methyl cellulose, polyvinylpyrrolidone, hydroxymethyl cellulose, hydroxyethyl cellulose and carboxymethyl cellulose.

In some embodiments, the water-insoluble cellulose derivative with bonded hydrophobic groups that can be used in the isolation layer of the present invention, the exemplary examples of its hydrophobic groups include, but are not limited to, methoxy, ethoxy, acetyl, butyryl and phthaloyl groups.

In some embodiments, the water-insoluble cellulose derivative with bonded hydrophobic groups that can be used in the isolation layer of the present invention, wherein the content of all hydrophobic groups is about 14% to 62% by weight relative to the water-insoluble cellulose derivative with bonded hydrophobic groups.

In some embodiments, the water-insoluble cellulose derivative with bonded hydrophobic groups that can be used in the isolation layer of the present invention, wherein the content of all hydrophobic groups is about 15% to 62% by weight, about 16% to 62% by weight, about 17% to 62% by weight, about 18% to 62% by weight, about 19% to 62% by weight, about 20% to 62% by weight, about 21% to 62% by weight, about 22% to 62% by weight, about 23% to 62% by weight, about 24% to 62% by weight, about 25% to 62% by weight, about 26% to 62% by weight, about 27% to 62% by weight, about 28% to 62% by weight, about 29% to 62% by weight, about 30% to 62% by weight, about 31% to 62% by weight, about 32% to 62% by weight, about 33% to 62% by weight, about 34% to 62% by weight, about 35% to 62% by weight, about 36% to 62% by weight, about 37% to 62% by weight, about 38% to 62% by weight, about 39% to 62% by weight, about 40% to 62% by weight, about 41% to 62% by weight, about 42% to 62% by weight, about 43% to 62% by weight, about 44% to 62% by weight, about 45% to 62% by weight, about 46% to 62% by weight, about 47% to 62% by weight, about 48% to 62% by weight, about 49% to 62% by weight, about 50% to 62% by weight, about 51% to 62% by weight, about 52% to 62% by weight, about 53% to 62% by weight, about 54% to 62% by weight, about 55% to 62% by weight, about 56% to 62% by weight, about 57% to 62% by weight, about 58% to 62% by weight, about 59% to 62% by weight, about 60% to 62% by weight or about 61% to 62% by weight relative to the water-insoluble cellulose derivative with bonded hydrophobic groups.

In some embodiments, the water-insoluble cellulose derivative with bonded hydrophobic groups that can be used in the isolation layer of the present invention, wherein the content of methoxy group, which acts as a hydrophobic group, is about 12% to 30% by weight relative to the water-insoluble cellulose derivative with bonded hydrophobic groups.

In some embodiments, the water-insoluble cellulose derivative with bonded hydrophobic groups that can be used in the isolation layer of the present invention, wherein the content of the methoxy group, which acts as a hydrophobic group, is about 12% to 13% by weight, about 12% to 14% by weight, about 12% to 15% by weight, about 12% to 16% by weight, about 12% to 17% by weight, about 12% to 18% by weight, about 12% to 19% by weight, about 12% to 20% by weight, about 12% to 21% by weight, about 12% to 22% by weight, about 12% to 23% by weight, about 12% to 24% by weight, about 12% to 25% by weight, about 12% to 26% by weight, about 12% to 27% by weight, about 12% to 28% by weight or about 12% to 29% by weight relative to the water-insoluble cellulose derivative with bonded hydrophobic groups.

In some embodiments, the water-insoluble cellulose derivative with bonded hydrophobic groups that can be used in the isolation layer of the present invention, wherein the content of the ethoxy group, which acts as a hydrophobic group, is about 28% to 55% by weight relative to the water-insoluble cellulose derivative with bonded hydrophobic groups.

In some embodiments, the water-insoluble cellulose derivative with bonded hydrophobic groups that can be used in the isolation layer of the present invention, wherein the content of the ethoxy group, which acts as a hydrophobic group, is about 28% to 29% by weight, about 28% to 30% by weight, about 28% to 31% by weight, about 28% to 32% by weight, about 28% to 33% by weight, about 28% to 34% by weight, about 28% to 35% by weight, about 28% to 36% by weight, about 28% to 37% by weight, about 28% to 38% by weight, about 28% to 39% by weight, about 28% to 40% by weight, about 28% to 41% by weight, about 28% to 42% by weight, about 28% to 43% by weight, about 28% to 44% by weight, about 28% to 45% by weight, about 28% to 46% by weight, about 28% to 47% by weight, about 28% to 48% by weight, about 28% to 49% by weight, about 28% to 50% by weight, about 28% to 51% by weight, about 28% to 52% by weight, about 28% to 53% by weight or about 28% to 54% by weight relative to the water-insoluble cellulose derivative with bonded hydrophobic groups.

In some embodiments, the water-insoluble cellulose derivative with bonded hydrophobic groups that can be used in the isolation layer of the present invention, wherein the content of the phthaloyl group, which acts as a hydrophobic group, is about 20% to 38% by weight relative to the water-insoluble cellulose derivative with bonded hydrophobic groups.

In some embodiments, the water-insoluble cellulose derivative with bonded hydrophobic groups that can be used in the isolation layer of the present invention, wherein the content of the phthaloyl group, which acts as a hydrophobic group, is about 20% to 21%, about 20% to 22% by weight, about 20% to 23% by weight, about 20% to 24% by weight, about 20% to 25% by weight, about 20% to 26% by weight, about 20% to 27% by weight, about 20% to 28% by weight, about 20% to 29% by weight, about 20% to 30% by weight, about 20% to 31% by weight, about 20% to 32% by weight, about 20% to 33% by weight, about 20% to 34% by weight, about 20% to 35% by weight, about 20% to 36% by weight or about 20% to 37% by weight relative to the water-insoluble cellulose derivative with bonded hydrophobic groups.

In some embodiments, the water-insoluble cellulose derivative with bonded hydrophobic groups that can be used in the isolation layer of the present invention, wherein the content of the acetyl group, which acts as a hydrophobic group, is about 2% to 48% by weight relative to the water-insoluble cellulose derivative with bonded hydrophobic groups.

In some embodiments, the water-insoluble cellulose derivative with bonded hydrophobic groups that can be used in the isolation layer of the present invention, wherein the content of the acetyl group, which acts as a hydrophobic group, is about 2% to 3% by weight, about 2% to 4% by weight, about 2% to 5% by weight, about 2% to 6% by weight, about 2% to 7% by weight, about 2% to 8% by weight, about 2% to 9% by weight, about 2% to 10% by weight, about 2% to 11% by weight, about 2% to 12% by weight, about 2% to 13% by weight, about 2% to 14% by weight, about 2% to 15% by weight, about 2% to 16% by weight, about 2% to 17% by weight, about 2% to 18% by weight, about 2% to 19% by weight, about 2% to 20% by weight, about 2% to 21% by weight, about 2% to 22% by weight, about 2% to 23% by weight, about 2% to 24% by weight, about 2% to 25% by weight, about 2% to 26% by weight, about 2% to 27% by weight, about 2% to 28% by weight, about 2% to 29% by weight, about 2% to 30% by weight, about 2% to 31% by weight, about 2% to 30 32% by weight, about 2% to 33% by weight, about 2% to 34% by weight, about 2% to 35% by weight, about 2% to 36% by weight, about 2% to 37% by weight, about 2% to 38% by weight, about 2% to 39% by weight, about 2% to 40% by weight, about 2% to 41% by weight, about 2% to 42% by weight, about 2% to 43% by weight, about 2% to 44% by weight, about 2% to 45% by weight, about 2% to 46% by weight or about 2% to 47% by weight relative to the water-insoluble cellulose derivative with bonded hydrophobic groups.

In some embodiments, the water-insoluble cellulose derivative with bonded hydrophobic groups that can be used in the isolation layer of the present invention, wherein the content of the butyryl group, which acts as a hydrophobic group, is about 14% to 22% by weight relative to the water-insoluble cellulose derivative with bonded hydrophobic groups.

In some embodiments, the water-insoluble cellulose derivative with bonded hydrophobic groups that can be used in the isolation layer of the present invention, wherein the content of the butyryl group, which acts as a hydrophobic group, is about 14% to 15% by weight, about 14% to 16% by weight, about 14% to 17% by weight, about 14% to 18% by weight, about 14% to 19% by weight, about 14% to 20% by weight or about 14% to 21% by weight relative to the water-insoluble cellulose derivative with bonded hydrophobic groups.

In some embodiments, exemplary examples of water-insoluble cellulose derivative with bonded hydrophobic groups that can be used in the isolation layer of the present invention include, but are not limited to, cellulose acetate, cellulose acetate phthalate, cellulose acetate butyrate, ethyl cellulose, hydroxypropyl methylcellulose phthalate, hydroxypropyl methylcellulose acetate succinate and carboxymethyl ethyl cellulose.

In some embodiments, exemplary examples of water-insoluble cellulose derivative with bonded hydrophobic groups that can be used in the isolation layer of the present invention include, but are not limited to, ethyl cellulose.

In some embodiments, ethyl cellulose that can be used in the present invention has a viscosity of 2 mPa·s to 120 mPa·s.

In some embodiments, ethyl cellulose that can be used in the present invention has a viscosity of about 2 mPa·s to 3 mPa·s, about 2 mPa·s to 4 mPa·s, about 2 mPa·s to 5 mPa·s, about 2 mPa·s to 5.5 mPa·s, about 2 mPa·s to 6 mPa·s, about 2 mPa·s to 7 mPa·s, about 2 mPa·s to 8 mPa·s, about 2 mPa·s to 9 mPa·s, about 2 mPa·s to 10 mPa·s, about 2 mPa·s to 11 mPa·s, about 2 mPa·s to 12 mPa·s, about 2 mPa·s to 13 mPa·s, about 2 mPa·s to 14 mPa·s, about 2 mPa·s to 15 mPa·s, about 2 mPa·s to 16 mPa·s, about 2 mPa·s to 17 mPa·s, about 2 mPa·s to 18 mPa·s, about 2 mPa·s to 19 mPa·s, about 2 mPa·s to 20 mPa·s, about 2 mPa·s to 21 mPa·s, about 2 mPa·s to 22 mPa·s, about 2 mPa·s to 23 mPa·s, about 2 mPa·s to 24 mPa·s, about 2 mPa·s to 25 mPa·s, about 2 mPa·s to 26 mPa·s, about 2 mPa·s to 27 mPa·s, about 2 mPa·s to 28 mPa·s, about 2 mPa·s to 29 mPa·s, about 2 mPa·s to 30 mPa·s, about 2 mPa·s to 32 mPa·s, about 2 mPa·s to 34 mPa·s, about 2 mPa·s to 36 mPa·s, about 2 mPa·s to 38 mPa·s, about 2 mPa·s to 40 mPa·s, about 2 mPa·s to 42 mPa·s, about 2 mPa·s to 44 mPa·s, about 2 mPa·s to 46 mPa·s, about 2 mPa·s to 48 mPa·s, about 2 mPa·s to 50 mPa·s, about 2 mPa·s to 60 mPa·s, about 2 mPa·s to 70 mPa·s, about 2 mPa·s to 80 mPa·s, about 2 mPa·s to 90 mPa·s, about 2 mPa·s to 100 mPa·s or about 2 mPa·s to 110 mPa·s.

In some embodiments, the molecular weight of ethyl cellulose that can be used in the present invention is about 30,000 g/mol to 220,000 g/mol.

In some embodiments, the molecular weight of ethyl cellulose that can be used in the present invention is about 30,000 g/mol to 35,000 g/mol, about 30,000 g/mol to 40,000 g/mol, about 30,000 g/mol to 45,000 g/mol, about 30,000 g/mol to 50,000 g/mol, about 30,000 g/mol to 55,000 g/mol, about 30,000 g/mol to 60,000 g/mol, about 30,000 g/mol to 65,000 g/mol, about 30,000 g/mol to 70,000 g/mol, about 30,000 g/mol to 75,000 g/mol, about 30,000 g/mol to 80,000 g/mol, about 30,000 g/mol to 85,000 g/mol, about 30,000 g/mol to 90,000 g/mol, about 30,000 g/mol to 95,000 g/mol, about 30,000 g/mol to 100,000 g/mol, about 30,000 g/mol to 105,000 g/mol, about 30,000 g/mol to 110,000 g/mol, about 30,000 g/mol to 115,000 g/mol, about 30,000 g/mol to 120,000 g/mol, about 30,000 g/mol to 125,000 g/mol, about 30,000 g/mol to 130,000 g/mol, about 30,000 g/mol to 135,000 g/mol, about 30,000 g/mol to 140,000 g/mol, about 30,000 g/mol to 145,000 g/mol, about 30,000 g/mol to 145,000 g/mol, about 30,000 g/mol to 150,000 g/mol, about 30,000 g/mol to 155,000 g/mol, about 30,000 g/mol to 160,000 g/mol, about 30,000 g/mol to 165,000 g/mol, about 30,000 g/mol to 170,000 g/mol, about 30,000 g/mol to 175,000 g/mol, about 30,000 g/mol to 180,000 g/mol, about 30,000 g/mol to 190,000 g/mol, about 30,000 g/mol to 195,000 g/mol, about 30,000 g/mol to 200,000 g/mol, about 30,000 g/mol to 205,000 g/mol, about 30,000 g/mol to 210,000 g/mol or about 30,000 g/mol to 215,000 g/mol.

In some embodiments, exemplary examples of water-insoluble cellulose derivative with bonded hydrophobic groups that can be used in the isolation layer of the present invention include, but are not limited to, cellulose acetate.

In some embodiments, the cellulose acetate that can be used in the present invention has a viscosity of about 120 mPa·s to 4,500 mPa·s, about 120 mPa·s to 3,800 mPa·s, about 120 mPa·s to 3,900 mPa·s, about 120 mPa·s to 4,000 mPa·s, about 120 mPa·s to 4,100 mPa·s, about 120 mPa·s to 4,200 mPa·s, about 120 mPa·s to 4,300 mPa·s or about 120 mPa·s to 4,400 mPa·s, as measured according to ASTMD1343 and D871 measurement methods.

In some embodiments, the cellulose acetate that can be used in the present invention has a viscosity of about 120 mPa·s to 330 mPa·s, about 130 mPa·s to 320 mPa·s, about 140 mPa·s to 310 mPa·s, about 150 mPa·s to 300 mPa·s, about 160 mPa·s to 290 mPa·s or about 170 mPa·s to 280 mPa·s, as measured according to ASTMD1343 and D871 measurement methods.

In some embodiments, the cellulose acetate that can be used in the present invention has a viscosity of about 3,000 mPa·s to 4,600 mPa·s, about 3,100 mPa·s to 4,500 mPa·s, about 3,200 mPa·s to 4,400 mPa·s, about 3,30015 mPa·s to 4,300 mPa·s, about 3,400 mPa·s to 4,200 mPa·s, about 3,500 mPa·s to 4,100 mPa·s, about 3,600 mPa·s to 4,000 mPa·s or about 3,700 mPa·s to 3,900 mPa·s, as measured according to ASTMD1343 and D871 measurement methods.

In some embodiments, the molecular weight of cellulose acetate that can be used in the present invention is about 10,000 g/mol to 45,000 g/mol, about 11,000 g/mol to 44,000 g/mol, about 12,000 g/mol to 43,000 g/mol, about 13,000 g/mol to 42,000 g/mol, about 14,000 g/mol to 41,000 g/mol, about 15,000 g/mol to 40,000 g/mol, about 16,000 g/mol to 39,000 g/mol, about 17,000 g/mol to 38,000 g/mol or about 18,000 g/mol to 37,000 g/mol.

In some embodiments, exemplary examples of water-insoluble cellulose derivative with bonded hydrophobic groups that can be used in the isolation layer of the present invention include, but are not limited to, cellulose acetate phthalate.

In some embodiments, the cellulose acetate phthalate that can be used in the present invention has a viscosity of about 35 mPa·s to 100 mPa·s, about 36 mPa·s to 99 mPa·s, about 37 mPa·s to 98 mPa·s, about 38 mPa·s to 97 mPa·s, about 39 mPa·s to 96 mPa·s, about 40 mPa·s to 95 mPa·s, about 41 mPa·s to 94 mPa·s, about 42 mPa·s to 93 mPa·s, about 43 mPa·s to 92 mPa·s, about 44 mPa·s to 91 mPa·s or about 45 mPa·s to 90 mPa·s, as measured using an Ubbelohde viscometer.

In some embodiments, the molecular weight of the cellulose acetate phthalate that can be used in the present invention is about 8,000 g/mol to 18,000 g/mol, about 9,000 g/mol to 17,000 g/mol, about 10,000 g/mol to 16,000 g/mol, about 11,000 g/mol to 15,000 g/mol or about 12,000 g/mol to 14,000 g/mol.

In some embodiments, exemplary examples of water-insoluble cellulose derivative with bonded hydrophobic groups that can be used in the isolation layer of the present invention include, but are not limited to, cellulose acetate butyrate.

In some embodiments, the cellulose acetate butyrate that can be used in the present invention has a viscosity of about 4,337 mPa·s to 7,137 mPa·s, about 4,537 mPa·s to 6,937 mPa·s, about 4,737 mPa·s to 6,737 mPa·s, about 4,937 mPa·s to 6,537 mPa·s, about 5,137 mPa·s to 6,337 mPa·s, about 5,337 mPa·s to 6,137 mPa·s or about 5,537 mPa·s to 5,937 mPa·s, as measured according to ASTMD1343 and D817 measurement methods.

In some embodiments, the molecular weight of the cellulose acetate butyrate that can be used in the present invention is about 60,000 g/mol to 70,000 g/mol, about 61,000 g/mol to 69,000 g/mol, about 62,000 g/mol to 68,000 g/mol, about 63,000 g/mol to 67,000 g/mol or about 64,000 g/mol to 66,000 g/mol.

In some embodiments, exemplary examples of water-insoluble cellulose derivative with bonded hydrophobic groups that can be used in the isolation layer of the present invention include, but are not limited to, hydroxypropyl methylcellulose phthalate.

In some embodiments, hydroxypropyl methylcellulose phthalate has a viscosity of about 20 mPa·s to 230 mPa·s, about 20 mPa·s to 25 mPa·s, about 20 mPa·s to 30 mPa·s, about 20 mPa·s to 35 mPa·s, about 20 mPa·s to 40 mPa·s, about 20 mPa·s to 45 mPa·s, about 20 mPa·s to 50 mPa·s, about 20 mPa·s to 55 mPa·s, about 20 mPa·s to 60 mPa·s, about 20 mPa·s to 65 mPa·s, about 20 mPa·s to 70 mPa·s, about 20 mPa·s to 75 mPa·s, about 20 mPa·s to 80 mPa·s, about 20 mPa·s to 85 mPa·s, about 20 mPa·s to 90 mPa·s, about 20 mPa·s to 95 mPa·s, about 20 mPa·s to 100 mPa·s, about 20 mPa·s to 105 mPa·s, about 20 mPa·s to 110 mPa·s, about 20 mPa·s to 115 mPa·s, about 20 mPa·s to 120 mPa·s, about 20 mPa·s to 125 mPa·s, about 20 mPa·s to 130 mPa·s, about 20 mPa·s to 135 mPa·s, about 20 mPa·s to 140 mPa·s, about 20 mPa·s to 145 mPa·s, about 20 mPa·s to 150 mPa·s, about 20 mPa·s to 155 mPa·s, about 20 mPa·s to 160 mPa·s, about 20 mPa·s to 165 mPa·s, about 20 mPa·s to 170 mPa·s, about 20 mPa·s to 175 mPa·s, about 20 mPa·s to 180 mPa·s, about 20 mPa·s to 185 mPa·s, about 20 mPa·s to 190 mPa·s, about 20 mPa·s to 195 mPa·s, about 20 mPa·s to 200 mPa·s, about 20 mPa·s to 205 mPa·s, about 20 mPa·s to 210 mPa·s, about 20 mPa·s to 215 mPa·s, about 20 mPa·s to 220 mPa·s or about 20 mPa·s to 225 mPa·s.

In some embodiments, the molecular weight of the hydroxypropyl methylcellulose phthalate that can be used in the present invention is about 30,000 g/mol to 160,000 g/mol, about 30,000 g/mol to 40,000 g/mol, about 30,000 g/mol to 50,000 g/mol, about 30,000 g/mol to 60,000 g/mol, about 30,000 g/mol to 70,000 g/mol, about 30,000 g/mol to 80,000 g/mol, about 30,000 g/mol to 90,000 g/mol, about 30,000 g/mol to 100,000 g/mol, about 30,000 g/mol to 110,000 g/mol, about 30,000 g/mol to 120,000 g/mol, about 30,000 g/mol to 130,000 g/mol, about 30,000 g/mol to 140,000 g/mol or about 30,000 g/mol to 150,000 g/mol.

In some embodiments, exemplary examples of water-insoluble cellulose derivative with bonded hydrophobic groups that can be used in the isolation layer of the present invention include, but are not limited to, hydroxypropyl methylcellulose acetate succinate.

In some embodiments, hydroxypropyl methylcellulose acetate succinate that can be used in the present invention has a viscosity of about 1.5 mPa·s to 10 mPa·s, about 1.5 mPa·s to 1.6 mPa·s, about 1.5 mPa·s to 1.8 mPa·s, about 1.5 mPa·s to 2 mPa·s, about 1.5 mPa·s to 2.2 mPa·s, about 1.5 mPa·s to 2.4 mPa·s, about 1.5 mPa·s to 2.6 mPa·s, about 1.5 mPa·s to 2.8 mPa·s, about 1.5 mPa·s to 3.0 mPa·s, about 1.5 mPa·s to 3.2 mPa·s, about 1.5 mPa·s to 3.4 mPa·s, about 1.5 mPa·s to 3.6 mPa·s, about 1.5 mPa·s to 3.8 mPa·s, about 1.5 mPa·s to 4 mPa·s, about 1.5 mPa·s to 4.5 mPa·s, about 1.5 mPa·s to 5 mPa·s, about 1.5 mPa·s to 5.5 mPa·s, about 1.5 mPa·s to 6 mPa·s, about 1.5 mPa·s to 6.5 mPa·s, about 1.5 mPa·s to 7 mPa·s, about 1.5 mPa·s to 7.5 mPa·s, about 1.5 mPa·s to 8 mPa·s, about 1.5 mPa·s to 8.5 mPa·s, about 1.5 mPa·s to 9 mPa·s or about 1.5 mPa·s to 9.5 mPa·s.

In some embodiments, the molecular weight of the hydroxypropyl methylcellulose acetate succinate that can be used in the present invention is about 14,500 g/mol to 23,500 g/mol, about 14,500 g/mol to 15,000 g/mol, about 14,500 g/mol to 15,500 g/mol, about 14,500 g/mol to 16,000 g/mol, about 14,500 g/mol to 16,500 g/mol, about 14,500 g/mol to 17,000 g/mol, about 14,500 g/mol to 17,500 g/mol, about 14,500 g/mol to 18,000 g/mol, about 14,500 g/mol to 18,500 g/mol, about 14,500 g/mol to 19,000 g/mol, about 14,500 g/mol to 19,500 g/mol, about 14,500 g/mol to 20,000 g/mol, about 14,500 g/mol to 20,500 g/mol, about 14,500 g/mol to 21,000 g/mol, about 14,500 g/mol to 21,500 g/mol, about 14,500 g/mol to 22,000 g/mol, about 14,500 g/mol to 22,500 g/mol or about 14,500 g/mol to 23,000 g/mol.

In some embodiments, exemplary examples of water-insoluble cellulose derivative with bonded hydrophobic groups that can be used in the isolation layer of the present invention include, but are not limited to, carboxymethyl ethyl cellulose.

In some embodiments, carboxymethyl ethyl cellulose that can be used in the present invention has a viscosity of about 5 mm²/s to 85 mm²/s, about 10 mm²/s to 80 mm²/s, about 15 mm²/s to 75 mm²/s or about 20 mm²/s to 70 mm²/s.

In some embodiments, the molecular weight of carboxymethyl ethyl cellulose that can be used in the present invention is about 44,000 g/mol to 54,000 g/mol, about 45,000 g/mol to 53,000 g/mol, about 46,000 g/mol to 52,000 g/mol, about 47,000 g/mol to 51,000 g/mol or about 48,000 g/mol to 50,000 g/mol.

In some embodiments, the weight ratio of the water-soluble polymer to the water-insoluble cellulose derivative with bonded hydrophobic groups in the isolation layer of the pellets of the present invention is about 1:3.

In some embodiments, the coating weight gain of the isolation layer of the pellets of the present invention relative to the weight of encapsulated core is about 0.5% to 13%.

In some embodiments, the coating weight gain of the isolation layer of the pellets of the present invention relative to the weight of encapsulated core is about 1.0% to 13%, about 1.5% to 13%, about 2.0% to 13%, about 2.5% to 13%, about 3.0% to 13%, about 3.5% to 13%, about 4.0% to 13%, about 4.5% to 13%, about 5% to 13%, about 5.5% to 13%, about 6% to 13%, about 6% to 13%, about 6.5% to 13%, about 7% to 13%, about 7.5% to 13%, about 8% to 13%, about 8.5% to 13%, about 9% to 13% or about 9.5% to 13%, about 10.5% to 13%, about 11% to 13%, about 11.5% to 13%, about 12% to 13%, about 12.5% to 13%.

In some embodiments, the coating weight gain of the isolation layer of the pellets of the present invention relative to the weight of encapsulated core is about 3% to 11%.

In some embodiments, the coating weight gain of the isolation layer of the pellets of the present invention relative to the weight of encapsulated core is about 4% to 10%.

In some embodiments, the weight of the cellulose derivative with bonded hydrophobic groups in the isolation layer of the pellets of the present invention is about 0.5% to 10% relative to the encapsulated core.

In some embodiments, the weight of the cellulose derivative with bonded hydrophobic groups in the isolation layer of the pellets of the present invention is about 1.0% to 10%, about 1.5% to 10%, about 2.0% to 10%, about 2.5% to 10%, about 3.0% to 10%, about 3.5% to 10%, about 4.0% to 10%, about 4.5% to 10%, about 5.0% to 10%, about 5.5% to 10%, about 6.0% to 10%, about 6.5% to 10%, about 7.0% to 10%, about 7.5% to 10%, about 8.0% to 10%, about 8.5% to 10%, about 9.0% to 10% or about 9.5% to 10% relative to the encapsulated core.

In some embodiments, the weight of the cellulose derivative with bonded hydrophobic groups in the isolation layer of the pellets of the present invention is about 2% to 8% relative to the encapsulated core.

In some embodiments, the weight of the cellulose derivative with bonded hydrophobic groups in the isolation layer of the pellets of the present invention is about 3% to 6% relative to the encapsulated core.

In some embodiments, the drug layer in the pellets of the present invention further comprises a water-soluble polymer.

In some embodiments, exemplary examples of water-soluble polymers that can be used in the drug layer of the present invention include, but are not limited to, hydroxypropyl cellulose, hydroxypropyl methyl cellulose, polyvinylpyrrolidone, hydroxymethyl cellulose, hydroxyethyl cellulose and carboxymethyl cellulose.

In some embodiments, the drug layer in the pellets of the present invention further comprises an anti-adherent. In some embodiments, exemplary examples of anti-adherent that can be used in the drug layer of the present invention include, but are not limited to, talc, colloidal silicon dioxide, titanium dioxide, magnesium stearate, stearic acid and glycerol monostearate.

In some embodiments, the drug layer in the pellets of the present invention further comprises a water-soluble polymer and an anti-adherent.

In some embodiments, the drug layer in the pellets of the present invention further comprises hydroxypropyl cellulose and talc.

In some embodiments, the pellets of the present invention exhibit optimized agglomeration performance, thereby reducing individual variability in drug administration, stabilizing the release of organic acids and drugs, enhancing bioavailability and reducing side effects.

In some embodiments, the pellets of the present invention can control the release rate of organic acids to achieve a slow release, thereby providing a more stable acidic microenvironment for drug release.

In another aspect, the present invention relates to a capsule comprising pellets of dabigatran etexilate or a pharmaceutically acceptable salt thereof, wherein the pellet comprises a pellet core, a drug layer containing dabigatran etexilate or a pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

In a further aspect, the present invention relates to a method for reducing individual variability of dabigatran etexilate or a pharmaceutically acceptable salt thereof, which includes administering a capsule comprising pellets of dabigatran etexilate or a pharmaceutically acceptable salt thereof, wherein the pellets comprise a pellet core, a drug layer containing dabigatran etexilate or a pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

In some embodiments, the individual is an adult patient with non-valvular atrial fibrillation.

In some embodiments, patients exhibit one or more of the following risk factors: previous stroke, transient ischemic attack or systemic embolism; left ventricular ejection fraction <40%; symptomatic heart failure ≥New York Heart Association Class II; age ≥75 years; age ≥65 years accompanied by any of the following diseases: diabetes mellitus, coronary artery disease or hypertension.

In yet another aspect, the present invention relates to a method for preventing stroke and systemic embolism in adult patients with non-valvular atrial fibrillation, which includes administering to patients in need thereof a prophylactically effective amount of capsule comprising pellets of dabigatran etexilate or a pharmaceutically acceptable salt thereof. Wherein, the pellet comprises a pellet core, a drug layer containing dabigatran etexilate or a pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

In some embodiments, patients exhibit one or more of the following risk factors: previous stroke, transient ischemic attack or systemic embolism; left ventricular ejection fraction <40%; symptomatic heart failure ≥New York Heart Association Class II; age ≥75 years; age ≥65 years accompanied by any of the following diseases: diabetes mellitus, coronary artery disease or hypertension.

In another aspect, the present invention relates to a method for improving the agglomeration of pellets in capsules, which includes providing the capsules with pellets of dabigatran etexilate or a pharmaceutically acceptable salt thereof, wherein the pellets comprise a pellet core, a drug layer containing dabigatran etexilate or a pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

In another aspect, a method for treating deep venous thrombosis and pulmonary embolism in adult patients, which includes administering to a patient in need thereof a prophylactically effective amount of capsule comprising pellets of dabigatran etexilate or a pharmaceutically acceptable salt thereof, wherein the pellet comprises a pellet core, a drug layer containing dabigatran etexilate or a pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

In yet another aspect, a method for reducing the recurrence risk of deep venous thrombosis and pulmonary embolism in adult patients, which includes administering to a patient in need thereof a prophylactically effective amount of capsule comprising pellets of dabigatran etexilate or a pharmaceutically acceptable salt thereof, wherein the pellet comprises a pellet core, a drug layer containing dabigatran etexilate or a pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

In another aspect, a method for preventing deep venous thrombosis and pulmonary embolism in adult patients after hip replacement surgery, which includes administering to a patient in need thereof a prophylactically effective amount of capsule comprising pellets of dabigatran etexilate or a pharmaceutically acceptable salt thereof, wherein the pellet comprises a pellet core, a drug layer containing dabigatran etexilate or a pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

In another aspect, a method for treating venous thromboembolic events in pediatric patients, which includes administering to a patient in need thereof a prophylactically effective amount of capsule comprising pellets of dabigatran etexilate or a pharmaceutically acceptable salt thereof. The pellet comprising a pellet core, a drug layer containing dabigatran etexilate or a pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

In yet another aspect, a method for reducing the recurrence risk of venous thromboembolic events in pediatric patients, which includes administering to a patient in need thereof a prophylactically effective amount of capsule comprising pellets of dabigatran etexilate or a pharmaceutically acceptable salt thereof, wherein the pellet comprises a pellet core, a drug layer containing dabigatran etexilate or a pharmaceutically acceptable salt thereof, and a protective layer, wherein the drug layer containing dabigatran etexilate or a pharmaceutically acceptable salt thereof is located between the pellet core and the protective layer, and the protective layer comprises a water-soluble polymer and a water-insoluble cellulose derivative with bonded hydrophobic groups.

In the following text, the present invention will be explained in detail through the following examples to better understand various aspects and advantages of the present invention. However, it should be understood that the following examples are non-limiting and are only used to illustrate some embodiments of the present invention.

### EXAMPLES

The reagents and equipment used in the examples of the present invention are conventional and commercially available. For example:

TABLE 1
Sources of raw materials and excipients

| Name of raw materials and | |
|---|---|
| excipients | Manufacturer |
| Dabigatran etexilate mesylate | Dr. Reddy's Laboratories Ltd |
| | MSN Laboratories Private Limited |
| Tartaric acid pellet core | International Product Services S.R.L |
| Hydroxypropyl methyl | Dow Group |
| cellulose | |
| Hydroxypropyl cellulose | Ashland Company Group |
| Talc | Imerys Talc Italy SpA |
| Ethyl cellulose | The Dow Chemical Company |
| Polyvinylpyrrolidone | BASF SE |
| Carboxymethyl ethyl cellulose | FREUND |
| Cellulose acetate phthalate | Eastman Chemical Company |
| Isopropanol | Guangdong Guanghua Technology Co., |
| | Ltd |

TABLE 2
Main experimental instruments and equipment

| Main instruments and | | |
|---|---|---|
| equipment | Manufacturer | Grade |
| Electronic balance | Sartorius Scientific Instruments (Beijing) Co., | BSA3238 |
| | Ltd | |
| Multi-functional | Chongqing Jinggong Pharmaceutical Machinery | DPL-II type |
| Pelletizing/Pill Making/ | Co., Ltd | |
| Coating Machine | | |
| Multi-functional | Chongqing Jinggong Pharmaceutical Machinery | DPL-0.2 |
| Pelletizing/Pill Making/ | Co., Ltd | |
| Coating Machine | | |
| Digital display top- | IKA (Guangzhou) Instrumentation Equipment | EURO. ST 40 KLCD |
| mounted mixer | Co., Ltd | SO25 |
| Dissolution tester | Tianda Tianfa Technology Co., Ltd | RC12AD |
| Ultraviolet-visible light | Shimadzu Instruments (Suzhou) Co., Ltd | UV-260 |
| Spectrophotometer | | |

[Examples 1 to 4 and Comparative Examples 1 to 2] Pellet capsule formulation
Formulation dosage unit: mg/capsule.

TABLE 3
Pellet capsule formulation for Examples 1 to 4 and Comparative Examples 1 to 2

| | | Example 1 | | Example 2 | | Example 3 | |
|---|---|---|---|---|---|---|---|
| | | Formulation | Percentage | Formulation | Percentage | Formulation | Percentage |
| Component | | dosage | % | dosage | % | dosage | % |
| Tartaric | Tartaric acid | 214.01 | 50.51 | 214.01 | 51.08 | 214.01 | 50.84 |
| acid layer | pellet core | | | | | | |
| | (25/30) | | | | | | |
| Isolation | Hydroxypropyl | 3.21 | 0.76 | 4.01 | 0.96 | 4.01 | 0.95 |
| layer | cellulose | | | | | | |
| | (HPC EXF) | | | | | | |
| | Ethyl cellulose | 9.63 | 2.27 | 12.04 | 2.87 | 12.04 | 2.86 |
| | (EC STD4) | | | | | | |
| Drug | Hydroxypropyl | 29.00 | 6.84 | 29.00 | 6.92 | 29.00 | 6.89 |
| layer | cellulose | | | | | | |
| | (HPC EXF) | | | | | | |
| | Dabigatran | 153.35 | 36.19 | 153.35 | 36.60 | 153.35 | 36.43 |
| | etexilate | | | | | | |
| | mesylate | | | | | | |
| | Talc | 2.05 | 0.48 | 2.05 | 0.49 | 2.05 | 0.49 |
| Protective | Hydroxypropyl | 6.61 | 1.52 | 2.32 | 0.55 | 3.35. | 0.80 |
| Layer | cellulose | | | | | | |
| | (HPC EXF) | | | | | | |
| | Talc | 0.64 | 0.15 | 0.22 | 0.05 | 0.32 | 0.08 |
| | Ethyl cellulose | 5.51 | 1.27 | 1.93 | 0.46 | 2.79 | 0.66 |
| | (EC STD4) | | | | | | |
| Total | | 424.00 | 100 | 418.94 | 100 | 420.93 | 100 |
| Weight ratio of HPC/EC | | 1.2 | | 1.2 | | 1.2 | |
| in the protective layer | | | | | | | |
| Weight gain of | | 3.10 | | 1.08 | | 1.56 | |
| protective layer relative | | | | | | | |
| to the weight of | | | | | | | |
| encapsulated core (%) | | | | | | | |
| Weight gain of EC in | | 1.34 | | 0.47 | | 0.67 | |
| protective layer relative | | | | | | | |
| to the weight of | | | | | | | |
| encapsulated core (%) | | | | | | | |
| Weight gain of HPC in | | 1.61 | | 0.56 | | 0.81 | |
| the protective layer | | | | | | | |
| relative to the weight of | | | | | | | |
| encapsulated core (%) | | | | | | | |
| | | | | Comparative | | Comparative | |
| | | Example 4 | | Example 1 | | Example 2 | |
| | | Formulation | Percentage | Formulation | Percentage | Formulation | Percentage |
| Component | | dosage | % | dosage | % | dosage | % |
| Tartaric | Tartaric acid | 214.01 | 49.70 | 214.01 | 51.41 | 214.01 | 49.05 |
| acid layer | pellet core | | | | | | |
| | (25/30) | | | | | | |
| Isolation | Hydroxypropyl | 3.21 | 0.75 | 4.01 | 0.96 | 3.21 | 0.74 |
| layer | cellulose | | | | | | |
| | (HPC EXF) | | | | | | |
| | Ethyl cellulose | 9.63 | 2.24 | 12.04 | 2.89 | 9.63 | 2.21 |
| | (EC STD4) | | | | | | |
| Drug | Hydroxypropyl | 29.00 | 6.74 | 29.00 | 6.97 | 29.00 | 6.65 |
| layer | cellulose | | | | | | |
| | (HPC EXF) | | | | | | |
| | Dabigatran | 153.35 | 35.61 | 153.35 | 36.84 | 153.35 | 35.15 |
| | etexilate | | | | | | |
| | mesylate | | | | | | |
| | Talc | 2.05 | 0.48 | 2.05 | 0.49 | 2.05 | 0.47 |
| Protective | Hydroxypropyl | 10.02 | 2.33 | 0.94 | 0.23 | 13.00 | 2.98 |
| Layer | cellulose | | | | | | |
| | (HPC EXF) | | | | | | |
| | Talc | 0.97 | 0.22 | 0.09 | 0.02 | 1.25 | 0.29 |
| | Ethyl cellulose | 8.35 | 1.94 | 0.79 | 0.19 | 10.83 | 2.48 |
| | (EC STD4) | | | | | | |

| Total | 430.58 | 100 | 416.28 | 100 | 436.34 | 100 |
|---|---|---|---|---|---|---|
| Weight ratio of HPC/EC | 1.2 | | 1.2 | | 1.2 | |
| in the protective layer | | | | | | |
| Weight gain of | 4.70 | | 0.44 | | 6.10 | |
| protective layer relative | | | | | | |
| to the weight of | | | | | | |
| encapsulated core (%) | | | | | | |
| Weight gain of EC in | 2.03 | | 0.19 | | 2.63 | |
| protective layer relative | | | | | | |
| to the weight of | | | | | | |
| encapsulated core (%) | | | | | | |
| Weight gain of HPC in | 2.44 | | 0.23 | | 3.16 | |
| the protective layer | | | | | | |
| relative to the weight of | | | | | | |
| encapsulated core (%) | | | | | | |

Note 1:
Solvents were removed during the process, and their usage was not counted in the intermediate product or final product.
Note 2:
The formulation dosage is the theoretical amount.

### [Examples 5 to 8 and Comparative Examples 3 to 4] Pellet Capsule Formulation

Formulation dosage unit: mg/capsule.

TABLE 4
Pellet capsule formulation for Examples 5 to 8 and Comparative Examples 3 to 4

| | | Example 5 | | Example 6 | | Example 7 | |
|---|---|---|---|---|---|---|---|
| | | Formulation | Percentage | Formulation | Percentage | Formulation | Percentage |
| Component | | dosage | % | dosage | % | dosage | % |
| Tartaric | Tartaric acid | 214.01 | 50.47 | 214.01 | 50.08 | 214.01 | 50.18 |
| acid layer | pellet core | | | | | | |
| | (25/30) | | | | | | |
| Isolation | Hydroxypropyl | 3.21 | 0.76 | 4.01 | 0.94 | 4.01 | 0.94 |
| layer | cellulose | | | | | | |
| | (HPC EXF) | | | | | | |
| | Ethyl cellulose | 9.63 | 2.27 | 12.04 | 2.82 | 12.04 | 2.82 |
| | (EC STD4) | | | | | | |
| Drug | Hydroxypropyl | 29.00 | 6.84 | 29.00 | 6.79 | 29.00 | 6.80 |
| layer | cellulose | | | | | | |
| | (HPC EXF) | | | | | | |
| | Dabigatran | 153.35 | 36.17 | 153.35 | 35.89 | 153.35 | 35.96 |
| | etexilate | | | | | | |
| | mesylate | | | | | | |
| | Talc | 2.05 | 0.48 | 2.05 | 0.48 | 2.05 | 0.48 |
| Protective | Hydroxypropyl | 4.86 | 1.15 | 6.10 | 1.43 | 6.45 | 1.51 |
| Layer | cellulose | | | | | | |
| | (HPC EXF) | | | | | | |
| | Talc | 0.64 | 0.15 | 0.64 | 0.15 | 0.60 | 0.14 |
| | Ethyl cellulose | 7.25 | 1.71 | 6.10 | 1.43 | 4.96 | 1.16 |
| | (EC STD4) | | | | | | |
| Total | | 424.00 | 100 | 427.31 | 100 | 426.48 | 100 |
| Weight ratio of HPC/EC | | 0.67 | | 1 | | 1.3 | |
| Weight gain of | | 3.10 | | 3.10 | | 2.90 | |
| protective layer relative | | | | | | | |
| to the weight of | | | | | | | |
| encapsulated core (%) | | | | | | | |
| Weight gain of EC in | | 1.76 | | 1.47 | | 1.20 | |
| protective layer relative | | | | | | | |
| to the weight of | | | | | | | |
| encapsulated core (%) | | | | | | | |
| Weight gain of HPC in | | 1.18 | | 1.47 | | 1.56 | |
| the protective layer | | | | | | | |
| relative to the weight of | | | | | | | |
| encapsulated core (%) | | | | | | | |
| | | | | Comparative | | Comparative | |
| | | Example 8 | | Example 3 | | Example 4 | |
| | | Formulation | Percentage | Formulation | Percentage | Formulation | Percentage |
| Component | | dosage | % | dosage | % | dosage | % |
| Tartaric | Tartaric acid | 214.01 | 50.42 | 214.01 | 50.18 | 214.01 | 50.52 |
| acid layer | pellet core | | | | | | |
| | (25/30) | | | | | | |
| Isolation | Hydroxypropyl | 4.01 | 0.94 | 4.01 | 0.94 | 3.21 | 0.76 |
| layer | cellulose | | | | | | |
| | (HPC EXF) | | | | | | |
| | Ethyl cellulose | 12.04 | 2.84 | 12.04 | 2.82 | 9.63 | 2.27 |
| | (EC STD4) | | | | | | |
| Drug | Hydroxypropyl | 27.18 | 6.41 | 29.00 | 6.80 | 29.00 | 6.85 |
| layer | cellulose | | | | | | |
| | (HPC EXF) | | | | | | |
| | Dabigatran | 153.35 | 36.13 | 153.35 | 35.96 | 153.35 | 36.20 |
| | etexilate | | | | | | |
| | mesylate | | | | | | |
| | Talc | 1.92 | 0.45 | 2.05 | 0.48 | 2.05 | 0.48 |
| Protective | Hydroxypropyl | 5.19 | 1.22 | 6.66 | 1.56 | 3.91 | 0.92 |
| Layer | cellulose | | | | | | |
| | (HPC EXF) | | | | | | |
| | Talc | 0.60 | 0.14 | 0.60 | 0.14 | 0.62 | 0.15 |
| | Ethyl cellulose | 6.18 | 1.46 | 4.76 | 1.12 | 7.81 | 1.84 |
| | (EC STD4) | | | | | | |

| Total | 424.47 | 100 | 426.48 | 100 | 423.59 | 100 |
|---|---|---|---|---|---|---|
| Weight ratio of HPC/EC | 0.84 | | 1.4 | | 0.5 | |
| Weight gain of | 2.90 | | 2.90 | | 3.00 | |
| protective layer relative | | | | | | |
| to the weight of | | | | | | |
| encapsulated core (%) | | | | | | |
| Weight gain of EC in | 1.50 | | 1.15 | | 1.90 | |
| protective layer relative | | | | | | |
| to the weight of | | | | | | |
| encapsulated core (%) | | | | | | |
| Weight gain of HPC in | 1.26 | | 1.61 | | 0.95 | |
| the protective layer | | | | | | |
| relative to the weight of | | | | | | |
| encapsulated core (%) | | | | | | |

Note 1:
Solvents were removed during the process, and their usage was not counted in the intermediate product or final product.
Note 2:
The formulation dosage is the theoretical amount.

### [Examples 9 to 11 and Comparative Examples 5 to 6] Pellet Capsule Formulation

Formulation dosage unit: mg/capsule.

TABLE 5
Pellet capsule formulation for Examples 9 to 11 and Comparative Examples 5 to 6

| | | Example 9 | | Example 10 | | Example 11 |
|---|---|---|---|---|---|---|
| | | Formulation | Percentage | Formulation | Percentage | Formulation |
| Component | | dosage | % | dosage | % | dosage |
| Tartaric | Tartaric acid | 214.01 | 50.38 | 214.01 | 50.67 | 214.01 |
| acid layer | pellet core | | | | | |
| | (25/30) | | | | | |
| Isolation | Hydroxypropyl | 3.21 | 0.76 | 4.01 | 0.95 | 3.21 |
| layer | cellulose | | | | | |
| | (HPC EXF) | | | | | |
| | Ethyl cellulose | 9.63 | 2.27 | 12.04 | 2.85 | 9.63 |
| | (EC STD4) | | | | | |
| Drug | Hydroxypropyl | 153.35 | 7.31 | 29.00 | 6.87 | 29.00 |
| layer | cellulose | | | | | |
| | (HPC EXF) | | | | | |
| | Dabigatran | 31.03 | 36.10 | 153.35 | 36.31 | 153.35 |
| | etexilate | | | | | |
| | mesylate | | | | | |
| | Talc | — | — | 2.05 | 0.49 | 2.05 |
| Protective | Hydroxypropyl | 7.03 | 1.66 | 4.08 | 0.97 | 8.10 |
| Layer | cellulose | | | | | |
| | (HPC EXF) | | | | | |
| | Talc | 0.68 | 0.16 | 0.39 | 0.09 | 0.78 |
| | Water-insoluble | 5.86 | 1.38 | 3.40 | 0.81 | 6.75 |
| | cellulose | | | | | |
| | derivative | | | | | |

| Total | 424.80 | 100 | 422.33 | 100 | 426.88 |
|---|---|---|---|---|---|
| Types or grades of water- | EC STD20 | | EC STD50 | | EC STD100 |
| insoluble cellulose | | | | | |
| derivatives in the | | | | | |
| protective layer | | | | | |
| Weight ratio of | 1.2 | | 1.0 | | 1.2 |
| HPC/water-insoluble | | | | | |
| cellulose derivatives in the | | | | | |
| protective layer | | | | | |
| Weight gain of protective | 3.30 | | 1.90 | | 3.80 |
| layer relative to the weight | | | | | |
| of encapsulated core (%) | | | | | |
| Weight gain of water- | 1.43 | | 0.82 | | 1.64 |
| insoluble cellulose | | | | | |
| derivatives in the | | | | | |
| protective layer relative to | | | | | |
| the weight of encapsulated | | | | | |
| core (%) | | | | | |
| Weight gain of HPC in the | 1.71 | | 0.98 | | 1.97 |
| protective layer relative to | | | | | |
| the weight of encapsulated | | | | | |
| core (%) | | | | | |

| | | | Comparative | | Comparative | |
|---|---|---|---|---|---|---|
| | | Example 11 | Example 5 | | Example 6 | |
| | | Percentage | Formulation | Percentage | Formulation | Percentage |
| Component | | % | dosage | % | dosage | % |
| Tartaric | Tartaric acid | 50.13 | 214.01 | 50.62 | 214.01 | 50.43 |
| acid layer | pellet core | | | | | |
| | (25/30) | | | | | |
| Isolation | Hydroxypropyl | 0.75 | 3.21 | 0.76 | 3.21 | 0.76 |
| layer | cellulose | | | | | |
| | (HPC EXF) | | | | | |
| | Ethyl cellulose | 2.26 | 9.63 | 2.28 | 9.63 | 2.27 |
| | (EC STD4) | | | | | |
| Drug | Hydroxypropyl | 6.79 | 29.00 | 6.86 | 153.35 | 7.31 |
| layer | cellulose | | | | | |
| | (HPC EXF) | | | | | |
| | Dabigatran | 35.92 | 153.35 | 36.27 | 31.03 | 36.13 |
| | etexilate | | | | | |
| | mesylate | | | | | |
| | Talc | 0.48 | 2.05 | 0.48 | — | — |
| Protective | Hydroxypropyl | 1.90 | 5.99 | 1.42 | 6.82 | 1.61 |
| Layer | cellulose | | | | | |
| | (HPC EXF) | | | | | |
| | Talc | 0.18 | 0.58 | 0.14 | 0.66 | 0.16 |
| | Water-insoluble | 1.58 | 4.99 | 1.18 | 5.68 | 1.34 |
| | cellulose | | | | | |
| | derivative | | | | | |

| Total | 100 | 422.81 | 100 | 424.39 | 100 |
|---|---|---|---|---|---|
| Types or grades of water- | EC STD100 | CMEC | | CAP | |
| insoluble cellulose | | | | | |
| derivatives in the | | | | | |
| protective layer | | | | | |
| Weight ratio of | 1.2 | 1.2 | | 1.2 | |
| HPC/water-insoluble | | | | | |
| cellulose derivatives in the | | | | | |
| protective layer | | | | | |
| Weight gain of protective | 3.80 | 2.81 | | 3.20 | |
| layer relative to the weight | | | | | |
| of encapsulated core (%) | | | | | |
| Weight gain of water- | 1.64 | 1.21 | | 1.38 | |
| insoluble cellulose | | | | | |
| derivatives in the | | | | | |
| protective layer relative to | | | | | |
| the weight of encapsulated | | | | | |
| core (%) | | | | | |
| Weight gain of HPC in the | 1.97 | 1.46 | | 1.66 | |
| protective layer relative to | | | | | |
| the weight of encapsulated | | | | | |
| core (%) | | | | | |

Note:
Solvents were removed during the process, and their usage was not counted in the intermediate product or final product.

The water-insoluble cellulose derivatives used in the present invention can be as follows:
Ethyl cellulose, grade STD4, with a viscosity of 3 mPa·s to 5.5 mPa·s; the hydrophobic group is ethoxy, with a substitution degree of 48.0% to 49.5%; and the weight-average molecular weight is less than 65,000 g/mol;
Ethyl cellulose, grade STD7, with a viscosity of 6 mPa·s to 8 mPa·s; the hydrophobic group is ethoxy, with a substitution degree of 48.0% to 49.5%; and the weight-average molecular weight is 65,000 g/mol;
Ethyl cellulose, grade STD10, with a viscosity of 9 mPa·s to 11 mPa·s; the hydrophobic group is ethoxy, with a substitution degree of 48.0% to 49.5%; and the weight-average molecular weight is 75,000 g/mol;
Ethyl cellulose, grade STD20, with a viscosity of 18 mPa·s to 22 mPa·s; the hydrophobic group is ethoxy, with a substitution degree of 48.0% to 49.5%; the weight-average molecular weight is 140,000 g/mol;
Ethyl cellulose, grade STD50, with a viscosity of 40 mPa·s to 55 mPa·s; the hydrophobic group is ethoxy, with a substitution degree of 48.0% to 49.5%; the weight-average molecular weight is 160,000 g/mol;
Ethyl cellulose, grade STD100, with a viscosity of 80 mPa·s to 110 mPa·s; the hydrophobic group is ethoxy, with a substitution degree of 48.0% to 49.5%; the weight-average molecular weight is 215,000 g/mol;
Carboxymethyl ethyl cellulose (CMEC), purchased from FREUND (Japan), wherein the substitution degree of ethoxy group, which acts as a hydrophobic group, is in a range from 32.5% to 43%; the weight-average molecular weight is 49,000 g/mol;
Cellulose acetate phthalate/cellulose acetate phthalate ester/cellulose acetate hydrogen phthalate (CAP), purchased from Eastman, the hydrophobic groups are acetyl and phthalyl (phthaloyl) groups, with a substitution degree of 51.5% to 62%. Wherein the substitution degree of acetyl groups is in a range from 21.5% to 26%, while the substitution degree of phthalyl (phthaloyl) groups is in a range from 30% to 36%. The number-average molecular weight is 13,000 g/mol.

### [Examples 12 to 16 and Reference Listed Drug] Pellet Capsule Formulation

Formulation dosage unit: mg/capsule.

The formulation composition of the commercially available Pradaxa® capsule (reference listed drug) is as follows: the tartaric acid layer contains acacia gum and tartaric acid, the isolation layer contains hydroxypropyl methyl cellulose, talc and dimethicone, and the drug layer contains dabigatran etexilate mesylate and hydroxypropyl cellulose. There is no protective layer.

TABLE 6
Pellet capsule formulation of Examples 12 to 16

| | | Example 12 | | Example 13 | | Example 14 |
|---|---|---|---|---|---|---|
| | | Formulation | Percentage | Formulation | Percentage | Formulation |
| Component | | dosage | % | dosage | % | dosage |
| Tartaric acid | Tartaric acid pellet | 214.01 | 50.97 | 214.01 | 50.11 | 214.01 |
| layer | core (25/30) | | | | | |
| Isolation | Hydroxypropyl | 3.21 | 0.76 | — | — | 4.01 |
| layer | cellulose (HPC | | | | | |
| | EXF) | | | | | |
| | Hydroxypropyl | — | — | 6.05 | 1.42 | — |
| | methyl cellulose | | | | | |
| | (HPMC E5) | | | | | |
| | Ethyl cellulose | 9.63 | 2.29 | — | — | 12.04 |
| | (EC STD4) | | | | | |
| | Talc | — | — | 6.05 | 1.42 | — |
| Drug layer | Hydroxypropyl | 29.00 | 6.91 | 31.03 | 7.27 | — |
| | cellulose (HPC | | | | | |
| | EXF) | | | | | |
| | Polyvinylpyrrolidone | — | — | — | — | 34.52 |
| | (PVP K30) | | | | | |
| | Dabigatran | 153.35 | 36.52 | 153.35 | 35.90 | 153.35 |
| | etexilate mesylate | | | | | |
| | Talc | 2.05 | 0.49 | 6.21 | 1.45 | 2.05 |
| Protective | Hydroxypropyl | 4.48 | 1.07 | 4.94 | 1.16 | 6.18 |
| Layer | cellulose (HPC | | | | | |
| | EXF) | | | | | |
| | Hydroxypropyl | — | — | — | — | — |
| | methyl cellulose | | | | | |
| | (HPMC E5) | | | | | |
| | Talc | 0.43 | 0.10 | 1.36 | 0.32 | 0.65 |
| | Ethyl cellulose | 3.73 | 0.89 | 4.12 | 0.96 | 6.18 |
| | (EC STD4) | | | | | |

| Total | 419.89 | 100 | 481.69 | 100 | 433.00 |
|---|---|---|---|---|---|
| Weight ratio of HPC/EC in the | 1.2 | | 1.2 | | 1.0 |
| protective layer | | | | | |
| Weight gain of protective layer | 2.10 | | 2.50 | | 3.10 |
| relative to the weight of | | | | | |
| encapsulated core (%) | | | | | |
| Weight gain of EC in protective | 0.91 | | 0.99 | | 1.47 |
| layer relative to the weight of | | | | | |
| encapsulated core (%) | | | | | |
| Weight gain of HPC in the | 1.09 | | 1.19 | | 1.47 |
| protective layer relative to the | | | | | |
| weight of encapsulated core (%) | | | | | |

| | | Example 14 | Example 15 | | Example 16 | |
|---|---|---|---|---|---|---|
| | | Percentage | Formulation | Percentage | Formulation | Percentage |
| Component | | % | dosage | % | dosage | % |
| Tartaric acid | Tartaric acid pellet | 49.42 | 214.01 | 50.38 | 214.01 | 49.50 |
| layer | core (25/30) | | | | | |
| Isolation | Hydroxypropyl | 0.93 | 3.21 | 0.76 | 4.01 | 0.93 |
| layer | cellulose (HPC | | | | | |
| | EXF) | | | | | |
| | Hydroxypropyl | — | — | — | — | — |
| | methyl cellulose | | | | | |
| | (HPMC E5) | | | | | |
| | Ethyl cellulose | 2.78 | 9.63 | 2.27 | 12.04 | 2.78 |
| | (EC STD4) | | | | | |
| | Talc | — | — | — | — | — |
| Drug layer | Hydroxypropyl | — | 29.00 | 6.83 | 27.18 | 6.29 |
| | cellulose (HPC | | | | | |
| | EXF) | | | | | |
| | Polyvinylpyrrolidone | 7.97 | — | — | — | — |
| | (PVP K30) | | | | | |
| | Dabigatran | 35.42 | 153.35 | 36.10 | 153.35 | 35.47 |
| | etexilate mesylate | | | | | |
| | Talc | 0.47 | 2.05 | 0.48 | 1.92 | 0.44 |
| Protective | Hydroxypropyl | 1.43 | — | — | 8.59 | 1.99 |
| Layer | cellulose (HPC | | | | | |
| | EXF) | | | | | |
| | Hydroxypropyl | — | 7.03 | 1.66 | — | — |
| | methyl cellulose | | | | | |
| | (HPMC E5) | | | | | |
| | Talc | 0.15 | 0.68 | 0.16 | 0.99 | 0.23 |
| | Ethyl cellulose | 1.43 | 5.86 | 1.38 | 10.22 | 2.36 |
| | (EC STD4) | | | | | |

| Total | 100 | 424.82 | 100 | 432.31 | 100 |
|---|---|---|---|---|---|
| Weight ratio of HPC/EC in the | 1.0 | 1.2 | | 0.84 | |
| protective layer | | | | | |
| Weight gain of protective layer | 3.10 | 3.30 | | 4.8 | |
| relative to the weight of | | | | | |
| encapsulated core (%) | | | | | |
| Weight gain of EC in protective | 1.47 | 1.43 | | 2.48 | |
| layer relative to the weight of | | | | | |
| encapsulated core (%) | | | | | |
| Weight gain of HPC in the | 1.47 | 1.71 | | 2.08 | |
| protective layer relative to the | | | | | |
| weight of encapsulated core (%) | | | | | |

Note 1:
Solvents were removed during the process, and their usage was not counted in the intermediate product or final product.
Note 2:
The formulation dosage is the theoretical amount.

### Preparation Method of Pellet Capsules

### 1) Isolation Layer Coating

1.1) Hydroxypropyl cellulose was weighed and dissolved in an aqueous solution of anhydrous ethanol. After dissolution, ethyl cellulose was added while stirring. Once fully dissolved, stirring was continued for no less than 15 minutes until fully clear solution is obtained (no white or translucent insoluble matter);

1.2) Tartaric acid pellets were placed into a fluidized bed, and the coating solution prepared in 1.1) was used for bottom-spray coating. The surface of the tartaric acid pellets was encapsulated with an isolation layer to obtain internally isolation layer pellets, which were then sieved;

### 2) Drug Layer Coating

2.1) Hydroxypropyl cellulose was dissolved in isopropanol solution, and dabigatran etexilate mesylate and talc were added. A high-shear disperser or mechanical stirrer was used to stir and disperse the mixture evenly;

2.2) The pellets in the internal isolation layer were placed into a bottom-spray fluidized bed and coated with the drug-containing suspension from 2.1). Once the target weight gain is achieved, spraying was stopped, and drying was continued using the fluidized bed for no less than 15 minutes to obtain drug-loaded pellets;

### 3) Protective Layer Coating

3.1) Hydroxypropyl cellulose was dissolved in an isopropanol solution. After completely dissolved, ethyl cellulose (the water-insoluble cellulose derivatives used in Comparative Examples 5 and 6) was then added for dissolution, followed by the addition of talc. Stirring was continued for no less than 15 minutes (the solvent used in this step for Comparative Example 5 was anhydrous ethanol, while the solvent used in this step for Comparative Example 6 was acetone);

3.2) The drug-loaded pellets were placed into a bottom-spray fluidized bed and coated with the coating solution from 3.1). When the target coating weight gain was achieved, spraying was stopped, and drying was continued for no less than 30 minutes to obtain the finished pellets.

### 4) Capsule Filling

4.1) The pellets were filled into 0 #capsule shells according to the loading capacity to obtain the pellet capsules.

### Testing Method for the Performance of Pellet Capsules

### 1. Screening Criteria for Release Experiment

The absorption segment of dabigatran etexilate in the human primarily occurs in the proximal small intestine. Premature release (before 10 minutes) will cause a large amount of active ingredient to be released in the stomach, leading to degradation of the active ingredient in gastric juice or a relatively low drug concentration when passing through the absorption segment. The ideal release profile involves a rapid and extensive release near the time when the preparation reaches the proximal small intestine, minimizing the risk of degradation of the active ingredient in gastric juice and ensuring a relatively high concentration of the active ingredient when passing through the absorption segment. Therefore, the cumulative release difference of the active ingredient between 10 minutes and 30 minutes (i.e., the cumulative release amount from the 10th minute to the 30th minute) was selected as the screening criterion for release.

### Release Rate Determination of Dabigatran Etexilate Capsules

Dissolution conditions: A rotation speed of 50 rpm, a water bath temperature of (37±0.5° C.), a dissolution medium of purified water, and a medium volume of 500 mL. Under the dissolution conditions, the samples to be tested were placed into the dissolution vessel. Samples of 10 mL solution withdrawn at 10 min, 15 min, 20 min, 30 min and 45 min, separately, filtered, and the initial filtrate of 2 mL was discarded to obtain the subsequent filtrate for the determination of dabigatran etexilate release. Three parallel measurements were taken for each sample, and the cumulative release of dabigatran etexilate at different release time points was calculated separately.

### 2. Screening Criteria for Agglomeration Experiment

Both the standard deviation (SD) of agglomeration and the weight of agglomerated pellets per 100 mg of pellets (in mg) were lower than those of the reference listed drug.

### Method for Agglomeration Experiment

The sample was placed into the settling basket, and immersed in 500 mL of purified water at a rotation speed of 35 rpm for 25 minutes, the basket was removed and dried. The pellets remaining in the basket were considered as agglomerated pellets. The weight of the agglomerated pellets was weighed and calculated, and parallel measurements were conducted on three samples.

### The Performance of Pellet Capsules

### 1. Cumulative Release

The cumulative release of pellet capsules prepared from different Examples and Comparative Examples are shown in Table 7.

TABLE 7
Cumulative release of pellet capsules prepared from
Examples 1 to 16 and Comparative Examples 1 to 6

| | | Time | | | | | |
|---|---|---|---|---|---|---|---|
| | | | | | | | (10-30) |
| | | 10 | 15 | 20 | 30 | 45 | Average |
| | | min | min | min | min | min | release |
| Group | | (%) | (%) | (%) | (%) | (%) | rate (%) |
| Example 1 | Cap1 | 8.21 | 26.86 | 45.67 | 72.15 | 87.27 | 61.54 |
| | Cap2 | 11.76 | 27.48 | 48.44 | 75.27 | 87.05 | |
| | Cap3 | 11.38 | 28.02 | 45.04 | 68.54 | 83.08 | |
| | Average | 10.45 | 27.45 | 46.38 | 71.99 | 85.80 | |
| | value | | | | | | |
| | % RSD | 18.64 | 2.12 | 3.90 | 4.68 | 2.75 | |
| Example 2 | Cap1 | 16.44 | 36.89 | 57.63 | 77.07 | 87.10 | 62.29 |
| | Cap2 | 8.86 | 27.69 | 47.37 | 72.19 | 86.97 | |
| | Cap3 | 14.96 | 32.70 | 50.60 | 77.86 | 83.29 | |
| | Average | 13.42 | 32.42 | 51.87 | 75.71 | 85.79 | |
| | value | | | | | | |
| | % RSD | 29.91 | 14.21 | 10.12 | 4.06 | 2.52 | |
| Example 3 | Cap1 | 12.19 | 33.18 | 53.22 | 77.35 | 87.03 | 63.60 |
| | Cap2 | 11.82 | 31.91 | 53.72 | 77.85 | 86.34 | |
| | Cap3 | 9.23 | 26.43 | 45.40 | 68.83 | 86.33 | |
| | Average | 11.08 | 30.51 | 50.78 | 74.68 | 86.57 | |
| | value | | | | | | |
| | % RSD | 14.53 | 11.77 | 9.19 | 6.78 | 0.46 | |
| Example 4 | Cap1 | 8.04 | 23.43 | 38.16 | 64.25 | 81.22 | 57.69 |
| | Cap2 | 6.39 | 22.14 | 38.98 | 65.41 | 82.05 | |
| | Cap3 | 10.23 | 26.69 | 43.35 | 68.07 | 81.18 | |
| | Average | 8.22 | 24.09 | 40.16 | 65.91 | 81.48 | |
| | value | | | | | | |
| | % RSD | 23.41 | 9.74 | 6.95 | 2.97 | 0.60 | |
| Example 5 | Cap1 | 12.39 | 26.55 | 40.24 | 59.82 | 77.43 | 50.83 |
| | Cap2 | 8.44 | 23.53 | 39.20 | 60.72 | 75.57 | |
| | Cap3 | 11.64 | 26.16 | 42.20 | 64.42 | 79.79 | |
| | Average | 10.82 | 25.41 | 40.55 | 61.65 | 77.60 | |
| | value | | | | | | |
| | % RSD | 19.34 | 6.47 | 3.76 | 3.96 | 2.73 | |
| Example 6 | Cap1 | 16.62 | 34.70 | 53.28 | 74.23 | 86.16 | 57.71 |
| | Cap2 | 15.89 | 34.86 | 52.57 | 75.23 | 85.65 | |
| | Cap3 | 15.16 | 32.88 | 50.06 | 71.35 | 83.78 | |
| | Average | 15.89 | 34.15 | 51.97 | 73.60 | 85.20 | |
| | value | | | | | | |
| | % RSD | 4.60 | 3.22 | 3.26 | 2.74 | 1.47 | |
| Example 7 | Cap1 | 3.14 | 24.68 | 42.58 | 69.32 | 81.55 | 61.79 |
| | Cap2 | 4.43 | 27.78 | 42.85 | 65.94 | 79.53 | |
| | Cap3 | 10.16 | 18.66 | 37.63 | 67.84 | 82.79 | |
| | Average | 5.91 | 23.71 | 41.02 | 67.70 | 81.29 | |
| | value | | | | | | |
| | % RSD | 63.20 | 19.55 | 7.16 | 2.50 | 2.03 | |
| Example 8 | Cap1 | 12.18 | 32.03 | 49.23 | 72.13 | 82.52 | 52.69 |
| | Cap2 | 14.87 | 34.71 | 50.37 | 67.54 | 79.08 | |
| | Cap3 | 21.32 | 39.41 | 51.62 | 66.78 | 78.15 | |
| | Average | 16.13 | 35.38 | 50.40 | 68.82 | 79.92 | |
| | value | | | | | | |
| | % RSD | 29.12 | 10.56 | 2.38 | 4.21 | 2.88 | |
| Example 9 | Cap1 | 18.52 | 34.47 | 47.14 | 68.56 | 83.88 | 56.93 |
| | Cap2 | 19.27 | 40.58 | 56.85 | 78.28 | 95.45 | |
| | Cap3 | 15.07 | 37.63 | 54.11 | 76.81 | 90.61 | |
| | Average | 17.62 | 37.56 | 52.70 | 74.55 | 89.98 | |
| | value | | | | | | |
| | % RSD | 12.71 | 8.14 | 9.50 | 7.02 | 6.46 | |
| Example 10 | Cap1 | 14.68 | 31.59 | 47.44 | 71.25 | 86.10 | 58.08 |
| | Cap2 | 14.86 | 33.39 | 52.77 | 74.67 | 88.52 | |
| | Cap3 | 17.07 | 35.05 | 53.37 | 74.93 | 87.93 | |
| | Average | 15.54 | 33.34 | 51.19 | 73.62 | 87.52 | |
| | value | | | | | | |
| | % RSD | 8.54 | 5.20 | 6.37 | 2.80 | 1.44 | |
| Example 11 | Cap1 | 8.60 | 21.06 | 34.14 | 56.70 | 74.99 | 49.86 |
| | Cap2 | 9.67 | 21.79 | 35.55 | 59.12 | 77.06 | |
| | Cap3 | 9.50 | 22.66 | 36.94 | 61.52 | 80.29 | |
| | Average | 9.26 | 21.84 | 35.54 | 59.12 | 77.45 | |
| | value | | | | | | |
| | % RSD | 6.22 | 3.66 | 3.94 | 4.08 | 3.45 | |
| Example 12 | Cap1 | 11.69 | 28.33 | 45.17 | 66.28 | 79.89 | 56.52 |
| | Cap2 | 11.69 | 29.59 | 47.12 | 71.33 | 82.25 | |
| | Cap3 | 18.99 | 36.36 | 53.19 | 74.31 | 85.56 | |
| | Average | 14.12 | 31.43 | 48.49 | 70.64 | 82.57 | |
| | value | | | | | | |
| | % RSD | 29.86 | 13.73 | 8.63 | 5.74 | 3.45 | |
| Example 13 | Cap1 | 20.71 | 57.38 | 70.98 | 82.59 | 90.11 | 60.58 |
| | Cap2 | 21.25 | 49.73 | 69.26 | 80.53 | 90.56 | |
| | Average | 20.98 | 53.55 | 70.12 | 81.56 | 90.33 | |
| | value | | | | | | |
| | % RSD | 1.84 | 10.09 | 1.73 | 1.78 | 0.35 | |
| Example 14 | Cap1 | 26.03 | 48.44 | 66.64 | 81.67 | 91.08 | 59.42 |
| | Cap2 | 17.10 | 43.52 | 59.23 | 77.75 | 89.90 | |
| | Cap3 | 23.80 | 53.86 | 66.89 | 85.76 | 93.80 | |
| | Average | 22.31 | 48.61 | 64.25 | 81.73 | 91.59 | |
| | value | | | | | | |
| Example 15 | Cap1 | 12.97 | 30.76 | 46.05 | 66.08 | 81.59 | 56.80 |
| | Cap2 | 14.64 | 32.97 | 49.87 | 73.03 | 84.79 | |
| | Cap3 | 12.23 | 30.02 | 47.80 | 71.14 | 85.11 | |
| | Average | 13.28 | 31.25 | 47.91 | 70.08 | 83.83 | |
| | value | | | | | | |
| | % RSD | 9.29 | 4.92 | 3.98 | 5.13 | 2.32 | |
| Example 16 | Cap1 | 25.44 | 45.28 | 60.94 | 76.60 | 87.97 | 53.66 |
| | Cap2 | 21.68 | 45.38 | 63.96 | 76.93 | 86.82 | |
| | Cap3 | 20.25 | 40.44 | 58.16 | 74.83 | 85.05 | |
| | Average | 22.46 | 43.70 | 61.02 | 76.12 | 86.61 | |
| | value | | | | | | |
| | % RSD | 11.95 | 6.47 | 4.76 | 1.48 | 1.70 | |
| Comparative | Cap1 | 4.31 | 14.79 | 29.73 | 56.34 | 81.61 | 47.69 |
| Example 2 | Cap2 | 2.44 | 10.16 | 22.22 | 51.83 | 79.89 | |
| | Cap3 | 4.50 | 15.16 | 30.82 | 46.15 | 83.32 | |
| | Average | 3.75 | 13.37 | 27.59 | 51.44 | 81.61 | |
| | value | | | | | | |
| | % RSD | 30.41 | 20.85 | 16.98 | 9.93 | 2.10 | |
| | Cap2 | 3.32 | 12.19 | 29.04 | 52.64 | 71.50 | |
| | Cap3 | 4.99 | 17.29 | 26.51 | 46.82 | 68.06 | |
| | Average | 3.51 | 13.28 | 26.76 | 49.32 | 69.71 | |
| | value | | | | | | |
| | % RSD | 39.74 | 27.05 | 8.10 | 6.07 | 2.48 | |
| Comparative | Cap1 | 6.77 | 17.45 | 31.53 | 53.26 | 69.86 | 46.51 |
| Example 4 | Cap2 | 7.71 | 18.76 | 32.65 | 57.39 | 71.57 | |
| | Cap3 | 9.40 | 23.95 | 32.97 | 52.67 | 70.23 | |
| | Average | 7.96 | 20.05 | 32.38 | 54.47 | 70.55 | |
| | value | | | | | | |
| | % RSD | 16.76 | 17.14 | 2.35 | 4.66 | 1.28 | |
| | Cap1 | 3.13 | 11.06 | 21.65 | 43.78 | 53.70 | 36.67 |
| Comparative | Cap2 | 3.86 | 9.27 | 18.28 | 38.68 | 55.52 | |
| Example 5 | Cap3 | 4.41 | 10.90 | 20.62 | 38.94 | 52.73 | |
| | Average | 3.80 | 10.41 | 20.18 | 40.47 | 53.99 | |
| | value | | | | | | |
| | % RSD | 16.99 | 9.52 | 8.57 | 7.10 | 2.62 | |
| Comparative | Cap1 | 0.75 | 4.06 | 13.13 | 39.70 | 49.15 | 37.03 |
| Example 6 | Cap2 | 0.67 | 5.30 | 15.96 | 39.29 | 47.22 | |
| | Cap3 | 0.52 | 4.35 | 12.70 | 34.05 | 45.50 | |
| | Average | 0.65 | 4.57 | 13.93 | 37.68 | 47.29 | |
| | value | | | | | | |
| | % RSD | 17.63 | 14.29 | 12.72 | 8.35 | 3.86 | |
| Reference | Cap1 | 10.84 | 41.90 | 60.06 | 73.90 | 81.19 | 47.84 |
| listed drug | Cap2 | 6.63 | 20.26 | 29.11 | 37.02 | 43.56 | |
| (Batch | Cap3 | 1.13 | 13.97 | 35.39 | 51.20 | 59.09 | |
| number: | Average | 6.20 | 25.38 | 41.52 | 54.04 | 61.28 | |
| 007760) | value | | | | | | |
| | % RSD | 78.49 | 57.73 | 39.40 | 34.43 | 30.86 | |

Note 1:
(10-30) Release rate = Cumulative release rate at 30 min − Cumulative release rate at 10 min.
Note 2:
The agglomeration performance test results for Comparative Example 1 and Comparative Example 3 showed severe agglomeration of the pellets, hence no release test was conducted.

### 2. Agglomeration Performance

The agglomeration performance of pellet capsules prepared in different Examples and Comparative Examples are shown in Table 8.

TABLE 8
Results of agglomeration experiments on pellet capsules prepared
in Examples 1 to 16 and Comparative Examples 1 to 6

| | | | | Agglomeration data | |
|---|---|---|---|---|---|
| | | | | | The weight of |
| | | | | | agglomerated |
| | Weight of agglomerated pellets | | | | pellet per 100 |
| | (mg) | | | SD | mg of pellets |
| Group | Cap1 | Cap2 | Cap3 | (%) | (mg) |
| Example 1 | 16.99 | 17.83 | 25.78 | 4.85 | 4.76 |
| Example 2 | 49.50 | 31.00 | 28.00 | 11.64 | 8.63 |
| Example 3 | 45.90 | 21.00 | 23.30 | 13.76 | 7.14 |
| Example 4 | 77.20 | 60.30 | 49.70 | 13.87 | 14.49 |
| Example 5 | 18.90 | 36.10 | 31.10 | 8.85 | 6.77 |
| Example 6 | 21.60 | 40.20 | 42.30 | 11.39 | 8.12 |
| Example 7 | 106.70 | 139.90 | 109.80 | 18.34 | 27.86 |
| Example 8 | 22.30 | 36.30 | 11.30 | 12.53 | 5.49 |
| Example 9 | 32.70 | 17.60 | 25.60 | 7.55 | 4.68 |
| Example 10 | 11.30 | 11.60 | 4.50 | 4.02 | 2.16 |
| Example 11 | 3.50 | 2.80 | 5.30 | 1.29 | 0.91 |
| Example 12 | 58.20 | 69.50 | 70.10 | 6.70 | 15.70 |
| Example 13 | 12.40 | 36.20 | 34.10 | 13.18 | 5.07 |
| Example 14 | 79.60 | 59.80 | 35.80 | 21.93 | 13.49 |
| Example 15 | 7.40 | 24.80 | 8.90 | 9.64 | 3.22 |
| Example 16 | 56.80 | 22.70 | 5.90 | 25.94 | 6.58 |
| Comparative | 201.80 | 229.90 | 214.60 | 14.07 | 51.75 |
| Example 1 | | | | | |
| Comparative | 79.70 | 128.80 | 85.70 | 26.78 | 22.47 |
| Example 2 | | | | | |
| Comparative | 401.20 | 410.10 | 406.70 | 4.49 | 95.20 |
| Example 3 | | | | | |
| Comparative | 15.80 | 1.70 | 1.00 | 8.35 | 1.46 |
| Example 4 | | | | | |
| Comparative | / | / | / | / | / |
| Example 5 | | | | | |
| Comparative | / | / | / | / | / |
| Example 6 | | | | | |
| Reference | 603.80 | 558.70 | 446.20 | 81.17 | 124.02 |
| listed drug | | | | | |
| (Batch | | | | | |
| number: | | | | | |
| 201730) | | | | | |

Note:
The release determination results for Comparative Example 5 and Comparative Example 6 showed slow release, hence no agglomeration performance test was conducted.

In the present invention, relational terms such as “first”, “second”, etc. are merely used to distinguish one entity or operation from another, and do not necessarily require or imply any actual relationship or order between these entities or operations.

From the foregoing, it can be understood that although specific embodiments of the present invention have been described for the purposes of exemplary explanation, various variants or improvements can be made by those skilled in the art without departing from the spirit and scope of the invention. These variants or modifications should fall within the scope of the claims appended to the present invention.

1. A pellet of dabigatran etexilate or a pharmaceutically acceptable salt thereof, comprising a pellet core, a drug layer containing dabigatran etexilate or pharmaceutically acceptable salt thereof, and a protective layer, wherein the drug layer containing dabigatran etexilate or pharmaceutically acceptable salt thereof is located between the pellet core and the protective layer, and the protective layer comprises a water-soluble polymer and a water-insoluble cellulose derivative with bonded hydrophobic groups; and
the weight ratio of the water-soluble polymer to the water-insoluble cellulose derivative with bonded hydrophobic groups is in a range from 0.67:1 to 1.3:1.

2. The pellet according to claim 1, wherein the hydrophobic group is selected from one or more of methoxy, ethoxy, acetyl, butyryl and phthaloyl groups, preferably from one or more of ethoxy, acetyl and butyryl groups; preferably, wherein the content of all hydrophobic groups is 14% to 62% by weight relative to the water-insoluble cellulose derivative with bonded hydrophobic groups.

3. The pellet according to claim 2, wherein the water-insoluble cellulose derivative with bonded hydrophobic groups is selected from cellulose acetate, cellulose acetate phthalate, cellulose acetate butyrate, ethyl cellulose, hydroxypropyl methylcellulose phthalate, hydroxypropyl methylcellulose acetate succinate and carboxymethyl ethyl cellulose; preferably ethyl cellulose.

4. The pellet according to claim 1, wherein the water-soluble polymer is selected from hydroxypropyl cellulose, hydroxypropyl methyl cellulose, polyvinylpyrrolidone, hydroxymethyl cellulose, hydroxyethyl cellulose and carboxymethyl cellulose; preferably from hydroxypropyl cellulose and hydroxypropyl methyl cellulose.

5. The pellet according to claim 1, wherein the coating weight gain of the protective layer relative to an encapsulated core is in a range from 1% to 5%.

6. The pellet according to claim 1, wherein the weight of the ethyl cellulose is in a range from 0.47% to 2.50% relative to an encapsulated core.

7. The pellet according to claim 1, wherein the weight of the water-soluble polymer is in a range from 0.56% to 2.44% relative to an encapsulated core.

8. The pellet according to claim 1, further comprising an isolation layer, wherein the isolation layer is located between the pellet core and the drug layer.

9. A capsule, comprising the pellet according to claim 1.

10. A method for improving the agglomeration of pellets in a dabigatran etexilate capsule, including providing the pellet according to claim 1 to the capsule.

11. A method for reducing individual variability of dabigatran etexilate or a pharmaceutically acceptable salt thereof, including administering the capsule according to claim 9.

12. A method for reducing the risk of stroke and systemic embolism in adult patients with non-valvular atrial fibrillation, including administering to a patient in need thereof a prophylactically effective amount of the capsule according to claim 9.

13. A method for treating deep venous thrombosis and pulmonary embolism in adult patients, including administering to a patient in need thereof a prophylactically effective amount of the capsule according to claim 9.

14. A method for reducing the recurrence risk of deep venous thrombosis and pulmonary embolism in adult patients, including administering to a patient in need thereof a prophylactically effective amount of the capsule according to claim 9.

15. A method for preventing deep venous thrombosis and pulmonary embolism in adult patients after hip replacement surgery, including administering to a patient in need thereof a prophylactically effective amount of the capsule according to claim 9.

16. A method for treating venous thromboembolic events in pediatric patients, including administering to a patient in need thereof a prophylactically effective amount of the capsule according to claim 9.

17. A method for reducing the recurrence risk of venous thromboembolic events in pediatric patients, including administering to a patient in need thereof a prophylactically effective amount of the capsule according to claim 9.
